(19) United States
(12) Patent Application Publication
(10) Pub. No.: US 2022/0211615 A1
(43) Pub. Date: Jul. 7, 2022

(54) FORMULATIONS AND METHODS FOR DRUG INSTILLATION INTO THE BLADDER AND TREATMENT OF BLADDER AILMENTS

(71) Applicant: TRIGONE PHARMA LTD., Ra'anana (IL)

(72) Inventors: Dan Moshe TOUITOU, Zichron Yaakov (IL); Elka TOUITOU, Zichron Yaakov (IL)

(21) Appl. No.: 17/606,033

(22) PCT Filed: Apr. 29, 2020
(86) PCT No.: PCT/IB2020/054044

§ 371 Date: Oct. 24, 2021

Related U.S. Application Data
(60) Provisional application No. 62/840,882, filed on Apr. 30, 2019.

(51) Int. Cl. A61K 9/00; A61K 47/10; A61K 31/05; A61K 31/426; A61K 31/135; A61K 31/216; A61K 38/48; A61K 31/167; A61K 31/4468; A61K 31/192; A61K 47/32; A61K 31/196; A61K 31/7048; A61K 31/704; A61K 31/407; A61K 31/496; A61K 31/7068; A61K 31/675; A61K 31/352; A61K 31/7036; A61K 31/573

(52) U.S. Cl. CPC A61K 9/0034; A61K 31/573; A61K 31/05; A61K 31/426; A61K 31/135; A61K 31/216; A61K 38/4893; A61K 31/167; A61K 31/4468; A61K 31/192; A61K 47/32; A61K 31/196; A61K 31/7048; A61K 31/704; A61K 31/407; A61K 31/496; A61K 31/7068; A61K 31/675; A61K 31/352; A61K 31/7036; A61K 47/10

(57) ABSTRACT

Polymeric liquid formulations for instillation into the bladder or kidney for prolonged release of an active agent and methods of treatment using the formulations are described. When in contact with urine, the formulation forms a mass in the bladder or kidney. The mass entraps the active agents and provides prolonged release of the active agents.

### CROSS-REFERENCE TO RELATED APPLICATION

The present application claims priority to U.S. Application No. 62/840,882, filed Apr. 30, 2019, the disclosure of which is incorporated herein by reference in its entirety.

### FIELD OF THE INVENTION

This invention generally relates to treating organs, such as the urinary tract, the bladder, and/or the kidneys.

### BACKGROUND OF THE INVENTION

The bladder is a muscular, hollow pelvic organ whose main functions include the storage and expulsion of urine. The relative impermeability of the bladder epithelium minimizes the systemic absorption of the drug and the side effects. The bladder is easily accessible using a catheter or cystoscope through the urethra by a simple procedure that can be performed by health care practitioners or even by patients themselves. However drugs or agents instilled intravesically into the bladder have limited efficacy due to periodic dilution and wash-out during urine formation and voiding. This decreases the time period that a drug is in contact with the targeted tissue. This method of delivery is cumbersome, as it requires repeated and frequent bladder catheterization and drug instillation.

Diseases of the urinary bladder, such as bladder carcinomas and interstitial cystitis, cause acute damage to the bladder wall and cannot be effectively treated by systemic administration of drugs. Such conditions may benefit from intra-bladder drug delivery (IBD), which involves direct instillation of drug into the bladder via a catheter, to attain high local concentrations of the drug with minimal systemic effects. IBD however has its limitations, since instilled drug solutions are diluted by urine and washed out of the bladder during voiding, necessitating repeated infusions of the drug.

Remaining challenges include issues regarding catheter or urethra obstruction during instillation, pain, patient tolerability during IBD of readymade matrixes, devices and solids. Thermoreversible solutions turn into gels in a temperature range close to human body temperature. Thus, their administration can be difficult, due to premature gelation of the solution before reaching the bladder and obstructing the catheter.

Other remaining challenges include high volume of the instilled drug formulation. The usual volume of drug formulation instilled is 50 ml, urging micturition and premature drug washout due to short retention time. Some strategies propose floating hydrogel to overcome the quick elimination of drug due to urine voiding. However, such gels need to acidulate the urine for floating or may obstruct the catheter due to their thermoreversible properties.

There remains a need for improved formulations and methods for instillation of active agents into the bladder and other organs containing urine, particularly which allow prolonged release of the active agents.

Therefore, it is an object of the present invention to provide formulations for instillation of active agents into the urinary tract, the bladder, and/or the kidneys.

It is another object of the present invention to provide methods for delivering active agents to the urinary tract, the bladder, and/or the kidneys.

It is a further object of the present invention to provide methods for treating or ameliorating one or more symptoms associated with a disorder or disease in or affecting a patient's urinary tract, bladder, and/or kidneys.

### SUMMARY OF THE INVENTION

Liquid formulations including an alcoholic solvent, one or more polymers, and one or more active agents for instillation into the urinary tract, the bladder, and/or the kidneys are described. Methods for treating or ameliorating one or more symptoms associated with a disorder or disease in or affecting a patient's urinary tract, bladder, and/or kidneys using a drug delivery system formed when the formulations contact urine in the urinary tract, the bladder, and/or the kidneys are also described.

The solvent includes one or more alcohols, such as one or more short-chain alcohols, such as ethanol and/or propylene glycol.

The formulation typically includes one or more polymers and one or more active agents. When in contact with urine, the one or more polymers precipitate from the formulation and entrap the one or more active agents, forming a mass (also referred to herein as “capture mass” or “capture”) in the urinary tract, the bladder, and/or the kidneys. The mass entraps the one or more active agents and releases the active agents over an extended period of time. The mass formed is not dependent on temperature; it typically floats on the surface of the urine or is immersed in the urine and does not obstruct the urethra, allowing for urine elimination. The release period and/or administrated dose of the active agents in the urinary tract, the bladder, and/or the kidneys can be modulated by the formulation composition, the volume instilled and the rate of instillation.

Typically, the one or more polymers are soluble in the alcoholic solvent and practically insoluble in water or an aqueous solution at a pH≤6. Optionally, the formulation includes one or more additives.

The method typically includes inserting a catheter, a cystoscope, or an ureteroscope into the urinary tract, the bladder, and/or the kidneys of the patient, and instilling the formulation into the urinary tract, the bladder, and/or the kidneys through the catheter, cystoscope, or ureteroscope. Following instillation, the formulation forms a mass in the patient's urine with the one or more active agents entrapped therein. Typically, the mass forms in the patient's urine instantaneously upon contact with urine. The release of the one or more active agents into the urine can begin immediately upon contact of the formulation with urine and continue for few hours up to several days, for example, up to 1 hour, up to 2 hours, up to 5 hours, up to 8 hours, up to 24 hours, up to 48 hours, up to 3 days, up to 4 days, up to 5 days, up to 6 days, up to 7 days, up to 15 days, or up to 30 days following instillation.

Optionally, the method includes repeating the step of instilling the same or a different formulation into the urinary tract, the bladder, and/or one or more kidneys every hour, every 2 hours, every 5 hours, every 8 hours, every day, every 2 days, every 3 days, every 5 days, every 7 days, every 10 days, every 30 days. Optionally, the method includes adjusting the release rate of the one or more active agents from the mass following the instillation of the formulation, for example, by instilling a basic solution into the urinary tract, the bladder, and/or the kidneys, administering bicarbonate medications, or consuming food that raises the pH of urine when it is digested, or a combination thereof.

In some embodiments, the method is effective to treat or ameliorate one or more symptoms associated with inflammation of the urinary tract, the bladder, and/or the kidneys and/or overactive bladder and/or neurogenic bladder. In some embodiments, the method is effective to kill or decrease the level of cancer cells associated with the bladder and/or kidney. In some embodiments, the method is effective to reduce the level of or prevent urinary tract, bladder, and/or kidney pain.

### BRIEF DESCRIPTION OF THE DRAWINGS

FIG. 1 is a photograph illustrating in situ formation of floating mass 10 after contact in a volume (30 ml) of urine 20. The largest dimension (d) of the mass 10 is approximately 6 cm.

FIG. 2A illustrates an exemplary mass 10′ formed in urine that can float on the surface of the urine. FIG. 2B illustrates an exemplary mass 10″ formed in urine that is immersed in the bulk of the urine.

FIG. 3 depicts an exemplary mass 10′″ after it is removed from urine.

### DETAILED DESCRIPTION OF THE INVENTION

Formulations and methods for intra-urinary tract, intra-bladder, and intra-kidney instillation are described herein. The liquid formulations described herein contain an alcoholic solution of one or more polymers and one or more active agents. When these formulations contact urine, a mass that entraps the one or more active agents forms immediately. The mass releases the active agent(s) over an extended period of time.

The liquid formulations disclosed herein can form a mass (also referred to herein as “capture” of “capture mass”) entrapping active agents instantaneously when in contact with urine. There is no lag time between instillation and the formation of the mass. In contrast to thermoreversible gels, the formed mass that acts as a prolonged delivery system that is not dependent on temperature. The formed mass can float on the surface of the urine or be immersed in the urine, and does not obstruct the urethra, i.e. it does not interfere with voiding and elimination of urine.

The administrated dose of active agents can be adjusted by adjusting the administration volume of the formulation. The methods described herein also allow for administrating different active agents by consecutive instillation through a catheter, a cystoscope, or an ureteroscope of formulations containing different active agents. The methods allow for the sequential or simultaneous release of different drugs into the urinary tract, bladder, and/or kidney(s).

### I. Formulations

Liquid formulations described herein include a solvent containing one or more alcohols, such as one or more short-chain alcohols, for example, ethanol and propylene glycol, one or more polymers, and one or more active agents. Optionally, the formulation also contains one or more additives. For example the formulation may contain a low concentration of water. For example, the formulation may contain water at a concentration less than 20% w/w of the formulation.

“Short-chain alcohols” generally refer to alcohols containing less than or equal to 4 carbon atoms. The composition contains one or more polymers and one or more active agents. When the liquid formulation contacts urine, the one or more polymers precipitate from the carrier into the urine, forming instantaneously in situ in the urinary tract, the bladder, or the kidney(s) a mass entrapping the one or more active agents. “Instantaneously” generally refers to within 5 seconds upon contact of the formulation with urine. For example, a mass is formed within 5 seconds, within 4 seconds, within 3 seconds, within 2 seconds, or within 1 second upon contact of the formulation with urine.

The mass in the urine of the urinary tract, the bladder, and/or the kidney(s) releases the active agent and provides prolonged release of the active agent to the urinary tract, the bladder, and/or the kidney(s). Release of the active agents, in the urine of the urinary tract, the bladder, or the kidney(s), from the mass can start immediately upon mass formation and continue for few hours up to several weeks. The delivery period in the urinary tract, the bladder, or the kidney(s) can be modulated by the formulation composition (i.e., polymer, active agents, additives, etc.), the volume instilled, and the frequency of repeated instillations.

The formulation can also include one or more additives, such as pharmaceutically acceptable excipients (e.g., surfactants) and/or one or more water- and alcohol-dispersible molecules, water- and alcohol-dispersible oligomers, or water- and alcohol-dispersible polymers, or a combination thereof.

A. Solvents

The formulation contains a solvent, typically an alcohol or a mixture of two or more alcohols that are miscible with each other. Typically, the alcohol is a short-chain alcohol. For example, the formulation contains ethanol or propylene glycol, or a combination thereof. Different purities of alcohol (e.g. absolute, or those containing low concentrations of water) may be used to form the formulation.

1. Alcohols

a. Short-Chain Alcohols

Typically, the one or more alcohols in the formulation are short-chain alcohols, such as C₁-C₄ alcohols, or C₂-C₄ alcohols, for example, ethanol and propylene glycol.

The alcohol may be a monohydric alcohol R—OH. R can be a saturated aliphatic hydrocarbon group containing 1-4 carbon atoms, such as 2-4 carbon atoms (i.e., C2-C4 alcohols). The saturated aliphatic hydrocarbon group can be linear, branched, or cyclic. The monohydric alcohol may be a primary alcohol, a secondary alcohol, or a tertiary alcohol.

Optionally, the alcohol is a primary alcohol having a linear saturated aliphatic hydrocarbon group, such as a methanol, ethanol, propanol, 1-butanol. For example, the alcohol can be ethanol.

Optionally, the alcohol is a primary alcohol having branched saturated aliphatic hydrocarbon group, such as isobutyl alcohol.

In some embodiments, the alcohol is a secondary alcohol, such as 2-propanol. In some embodiments, the alcohol is a tertiary alcohol, such as a 2-methyl-2-propanol.

Optionally, the alcohol contains more than one hydroxyl group. For example, the alcohol can be a diol, a triol, or a tetraol, etc. For example, the alcohol can be ethane-1,2-diol, propane-1,2-diol, propane-1,2,3-triol, or butane-1,2,3,4-tetraol. For example, the alcohol can be glycol (i.e., an aliphatic diol), such as propylene glycol.

b. Concentrations of Alcohols

Generally, the total concentration of the one or more alcohols in the formulation is from 10% to 90% w/w, from 12% to 90% w/w, from 12% to 50% w/w, from 20% to 80% w/w, from 30% to 75% w/w, from 40% to 90% w/w, from 50% to 95% w/w, or from 60% to 98% w/w of the formulation. The term “total concentration of the one or more alcohols” refers to the total weight of the one or more alcohols relative to the total weight of the formulation.

Optionally, the one or more alcohols contain ethanol and the concentration of ethanol is up to 49% w/w, up to 35% w/w, from 12% to 35% w/w, from 12% to 90% w/w, from 20% to 80% w/w, from 30% to 75% w/w, from 40% to 90% w/w, from 50% to 95% w/w, or from 60% to 98% w/w of the formulation. The ethanol can be ethanol absolute or ethanol having a different purity level.

Optionally, the one or more alcohols contain a glycol and the concentration of glycol is up to 96% w/w, from 12% to 96% w/w, from 12% to 45% w/w, from 20% to 60% w/w, from 1% to 55% w/w of the formulation.

Optionally, the one or more alcohols contain propylene glycol and the concentration of propylene glycol is up to 96% w/w, from 12% to 96% w/w, from 33% to 95%, from 12% up to 45% w/w, from 20% to 60% w/w, from 1% to 55% w/w, from 1% to 10% w/w, from 2% to 8% w/w, or from 2% to 7% w/w of the formulation.

When the formulation contains two or more miscible alcohols, the concentration of each alcohol can be in a suitable range to provide a total concentration of the one or more alcohols of 45% to 98% w/w, from 45% to 95% w/w, from 45% to 90% w/w, from 40% to 85% w/w, from 45% to 80% w/w, 50% to 98% w/w, from 50% to 95% w/w, from 50% to 90% w/w, from 50% to 85% w/w, from 50% to 80% w/w, from 60% to 98% w/w, from 60% to 95% w/w, from 60% to 90% w/w, from 60% to 85% w/w, or from 60% to 80% w/w of the formulation. For example, the concentration of a first alcohol can be in the range of 1% to 11% w/w, 1% to 12% w/w, 2% to 10% w/w, 2% to 15% w/w, 2% to 20% w/w, 1% to 89% w/w, 10% to 40% w/w, 5% to 45% w/w, 5% to 15% w/w, 20% to 60% w/w, 30% to 60% w/w, 15% to 60% w/w, 5% to 35% w/w, 10% to 80% w/w, 5% to 85% w/w, 5% to 90% w/w, 10% to 85% w/w, 10% to 50% w/w, 5% to 55% w/w, 1% to 97% w/w, 1% to 89% w/w, 12% to 50% w/w, 1% to 19% w/w, or 12% to 85% w/w of the formulation; and the concentration of the second alcohol can be in the range of 1% to 10% w/w, 1% to 15% w/w, 1% to 20% w/w, 1% to 25% w/w, 1% to 30% w/w, 1% to 35% w/w, 1% to 40% w/w, 1% to 45% w/w, 1% to 50% w/w, 1% to 55% w/w, 1% to 96% w/w, 2% to 8% w/w, 2% to 7%, 2% to 10% w/w, 2% to 15% w/w, 2% to 20^{%} w/w, 10% to 96% w/w, 10% to 90% w/w, 10% to 85% w/w, 10% to 80% w/w, 10% to 75% w/w, 20% to 45% w/w, 20% to 50% w/w, 20% to 60% w/w, or 20% to 65% w/w of the formulation.

The total concentration ranges described above for the one or more alcohols in the formulation refer to the concentration of the one or more alcohols having a purity of >99% v/v (the alcohol absolute) in the formulation. However, alcohols having different purities may be present in the formulation; however the concentration of water in such alcohols is not included in the total concentration ranges described above. Each of the one or more alcohols in the formulation can be an alcohol absolute (i.e., having an alcohol purity >99% v/v) or an alcohol having a purity of from 80% to 99% v/v. For example, the alcohol can be ethanol absolute (i.e., having purity >99% v/v) or an ethanol having a purity of from 80% to 99% v/v. For example, the alcohol can be glycol absolute (i.e., having an alcohol purity >99% v/v) or a glycol having a purity of from 80% to 99% v/v. For example, the alcohol can be propylene glycol absolute, such as a propylene glycol that contains less than 0.5% v/v water, such as from 0.2% to 0.5% v/v water, see, for example, U.S. Pharmacopeia.

When an alcohol having a purity of from 80% to 99% v/v is used, the formulation can contain a low concentration of water, depending on the purity of the alcohol(s). For example, the formulation may contain water at a concentration less than 20% w/w of the formulation, such as from 0.1% to 20% w/w, from 0.5% to 20% w/w, from 0.5% to 10% w/w, from 0.5% to 5% w/w, from 0.5% to 5% w/w, from 1% to 20% w/w, from 1% to 15% w/w, or from 1% to 10% w/w of the formulation.

B. Polymers

The liquid formulation includes one or more polymers.

1. Polymer Properties

The one or more polymers are soluble in alcohols, such as ethanol and/or propylene glycol, and practically insoluble in water and/or an aqueous solution at pH 6.0 or lower. When in contact with urine, the one or more polymers precipitate from the formulation and form a mass that entraps the one or more active agents spontaneously in the urine. Typically, the one or more polymers form the mass instantaneously (i.e., less than about 1 second upon contact with urine) in situ in urine and incorporate the one or more active agents in the formed mass.

Generally, the polymers have a solubility in the one or more alcohols of the formulation, such as ethanol and/or propylene glycol, of at least 1 g polymer/100 mL of the alcohol at room temperature (R.T.).

Generally, the polymers are practically insoluble in water or an aqueous at pH 6.0 or lower. Such polymers can also be characterized as insoluble in water or an aqueous at pH 6.0 or lower. Polymers that are practically insoluble have a solubility of 1 g or less of the polymer/10,000 mL of water or the aqueous solution at R.T., see, for example, U.S. Pharmacopeia, “Description and Solubility.” Generally, “room temperature” refers to a temperature between about 20° C. and about 25° C. under atmospheric pressure.

One or more of the polymers can be anionic, cationic, or nonionic polymers.

2. Exemplary Polymers

Exemplary polymers include, but are not limited to, ethylcellulose and acrylate polymers. Any other suitable polymers can be used in the formulations as long as the polymer is soluble in alcohols, particularly the one or more alcohols, and practically insoluble in water and/or an aqueous solution at pH 6.0 or lower. For example, ethylcellulose, polyacrylates, polycarbophil, and any polymers that are soluble in an alcohol and practically insoluble in water or an aqueous solution with pH≤6. For example, the formulation could contain a polymer that is at least as soluble as Eudragit® S100 in an alcohol and at least as insoluble as Eudragit® S100_{[TW1][DT2][RM3][DT4]} in water or an aqueous solution with pH≤6.

a. Ethylcellulose

Optionally the formulation contains ethylcellulose. Generally, ethylcellulose can increase the floating property of the formed mass in urine.

Typically, the ethylcellulose has an ethoxyl content from 40 wt % to 49.5 wt %, from 40 wt % to 49 wt %, from 45 wt % to 49.5 wt %, from 45 wt % to 47 wt %, from 48 wt % to 49.5 wt %, or from 46 wt % to 48 wt % of the ethylcellulose (i.e., the weight of the ethoxyl groups relative to the total weight of the ethylcellulose), for example, from 46 wt % to 48 wt % of the ethylcellulose.

Suitable ethylcelluloses include, but are not limited to, Ethocel™ polymers such as Ethocel™ med. 100.

The physical and chemical properties of Ethocel™ polymers are known, see, for example, “Ethylcellulose polymers technical handbook” by Dow Chemical Company, 2005. For example, Ethocel™ std. 100 is soluble in ethanol.

b. Acrylate Polymers

Optionally the formulation contains one or more acrylate polymers. Generally, the acrylate polymers can be anionic, cationic, or nonionic polymers.

Suitable acrylate polymers include, but are not limited to, poly(methacrylates) and copolymers thereof, poly(ethacrylates) and copolymers thereof, and copolymers of poly(methacrylates) and poly(ethacrylates), for example, poly(methyl methacrylate), poly(ethyl methacrylate), poly(N,N-dimethylaminoethyl methacrylate), poly(butyl methacrylate), or poly(ethylacrylate), or a copolymer thereof, or acrylic acid polymers crosslinked with divinyl glycol.

Known poly(methacrylates), poly(ethacrylates), their copolymers, and acrylic acid polymers crosslinked with divinyl glycol can be included in the formulations, such as, for example, Eudragit® polymers, Acryl-EZE®, and polycarbophil.

Suitable anionic Eudragit® polymers include, but are not limited to, Eudragit® S100, Eudragit® L100, and Eudragit® E100, or a combination thereof. Suitable cationic Eudragit® polymers include, but are not limited to, Eudragit® RS, and Eudragit® RL, or a combination thereof. Suitable nonionic Eudragit® polymers include, but are not limited to, Eudragit® NE/NM which is non-dependent on pH, and a combination of a cationic polymer and anionic polymer described above. Suitable acrylic acid polymers crosslinked with divinyl glycol include Polycarbophil.

The physical and chemical properties of Eudragit® polymers are known, see, for example, “Eudragit®—setting benchmarks in oral solid dosage forms since 1954” brochure by Evonik Industries. For example, Eudragit® S100 is soluble at pH 7 or higher and Eudragit® L100 is soluble at pH 6.5 and higher.

3. Concentrations of Polymers

Generally, the total concentration of the one or more polymers in the formulation is in a range from 1 wt % to 20 wt %, from 2 wt % to 20 wt %, from 1 wt % to 10 wt %, from 2 wt % to 8 wt %, or from 2 wt % to 7 wt % of the formulation. The term “total concentration of the one or more polymers” refers to the total weight of the one or more polymers relative to the total weight of the formulation.

Optionally, the one or more polymers include one or more ethylcelluloses and the total concentration of the ethylcellulose is from 1% to 10% w/w, from 2% to 8% w/w, or from 2% to 7% w/w of the formulation.

Optionally, the one or more polymers include one or more polymethacrylates and the total concentration of the one or more polymethacrylates is from 1% to 10% w/w, from 2% to 8% w/w, or from 2% to 7% w/w of the formulation.

Optionally, the one or more polymers include one or more polyethacrylates and the total concentration of the one or more polyethacrylates is from 1% to 10% w/w, from 2% to 8% w/w, or from 2% to 7% w/w of the formulation.

Optionally, the one or more polymers include a Eudragit® polymer and the concentration of the Eudragit® polymer is from 1% to 10% w/w, from 2% to 8% w/w, or from 2% to 7% w/w of the formulation.

Optionally, the one or more polymers include more than one Eudragit® polymer and the total concentration of the Eudragit® polymers is from 1% to 10% w/w, from 2% to 8% w/w, or from 2% to 7% w/w of the formulation. By adjusting the polymer concentration the release profile can be modulated. For example, increasing the concertation of Eudragit® S100 can increase the time period of the release of the one or more active agents and reduce the erosion rate of the mass.

When the formulation contains two or more polymers, the concentration of each polymer can be in a suitable range to provide a total concentration of the one or more polymers from 2% to 20% w/w, from 2% to 10% w/w, from 2% to 8% w/w, from 2% to 7% w/w, from 5% to 20% w/w, from 5% to 18% w/w, from 5% to 15% w/w, from 5% to 12% w/w, or from 5% to 10% w/w of the formulation. For example, the concentration of a first polymer can be in the range of 0.5% to 19.5% w/w, 0.5% to 15% w/w, 0.5% to 10% w/w, 1% to 19% w/w, 1% to 15% w/w, 1% to 10% w/w, 1% to 9% w/w, 1% to 8% w/w, 1% to 5% w/w, 2% to 18% w/w, from 2% to 15% w/w, from 2% to 10% w/w, from 2% to 8% w/w, or from 2% to 7% w/w, from 5% to 15% w/w, or from 5% to 10% w/w of the formulation; and the concentration of the second polymer can be 0.5% to 19.5% w/w, 0.5% to 15% w/w, 0.5% to 10% w/w, 1% to 19% w/w, 1% to 15% w/w, 1% to 10% w/w, 1% to 9% w/w, 1% to 8% w/w, 1% to 5% w/w, 2% to 18% w/w, from 2% to 15% w/w, from 2% to 10% w/w, from 2% to 8% w/w, or from 2% to 7% w/w, from 5% to 15% w/w, or from 5% to 10% w/w of the formulation.

C. Active Agents

The active agents in the formulation can be any suitable therapeutic, prophylactic, or diagnostic agents, or a combination thereof. In particular, the active agents are suitable for treating or ameliorating one or more symptoms associated with a disorder or disease in or affecting the urinary tract, the bladder, and/or the kidney(s).

1. Properties

The active agents in the formulation can be hydrophilic, amphiphilic, or lipophilic. Optionally, the formulation contains a combination of such active agents.

Typically, lipophilic and amphiphilic active agents that are soluble in the alcohol(s) can be dissolved in the formulation and hydrophilic active agents that are insoluble in the alcohols can be dispersed in the formulation.

Suitable active agents can be present in the formulation in either a pharmaceutical salt form, a base drug form, or a combination of both the pharmaceutical form and the base form.

In some embodiments, the formulation contains a water-soluble form of a drug, such as a pharmaceutical salt of a drug.

In some embodiments, the formulation an alcohol-soluble form of the drug (e.g., a base form of the drug).

In some embodiments, the formulation contains a combination of the pharmaceutical salt form and the base form of the drug.

For example, lidocaine can be present in the formulation as a base form that is soluble in alcohol and a pharmaceutical form (e.g., lidocaine HCl) that is soluble in water.

For example, local anesthetic drugs can also be present in the formulation in both forms, base form and pharmaceutical salt form, where the pharmaceutical salt form is soluble in water and alcohol and the base form is soluble in an alcohol, such as ethanol, and slightly soluble in water (i.e., having a solubility of from 1 g drug/100 mL of water to 1 g drug/10,000 mL of water at room temperature (R.T.)). In such cases, when the mass (or “capture”) is formed in the urine, the water-soluble form is released more quickly while the alcohol-soluble form is released in the urine over an extended period of time. This permits a drug release profile with a quick onset and a prolonged release.

2. Exemplary Active Agents

The active agents can be selected from various therapeutic classes such as antinociceptic, anesthetic, anti-inflammatory, antibiotic, muscarinic, mitotic, antimitotic, chemotherapic; probes for diagnosis; pH buffering agents; and radioactive isotopes, and a combination thereof.

Exemplary active agents include, but are not limited to, antibiotics, cannabinoids, anti-spastics, drugs used in the treatment of interstitial cystitis, urinary tract infection, bladder pain syndrome (BPS), and/or bladder cancer, analgesics, anti-bacterials, antimicotic, antifungals, antihistamines, anti-inflammatories, antineoplastics, antivirals, corticosteroids, cytotoxics, decongestants, diuretics, hormones, immune-suppressives, muscle relaxants, and sex hormones, and a combination thereof.

Optionally, the active agents include one or more drugs. The drug may be a small molecule drug (i.e., a small molecule having a molecular weight less than 900 Da), a biological agent (e.g., DNA, RNA, plasmids, a protein, or an antibody), a metabolite, or a radioactive molecule, or a combination thereof. The drug may be in its pharmaceutical salt form, hydrate form, free acid form, and/or free base form.

Optionally, the active agents include a single drug, or more than one drug from a single therapeutic class, or two or more drugs from different therapeutic classes. For example, the active agents include anticholinergic drugs, such as oxybutynin; PDE5 inhibitors, such as sildenafil and tadolafil; antinociceptic drugs and antinflamatory drugs, such as lidocaine and cannabidiol; phentanyl and cannabidiol; paracetamol, ibuprofen, and cannabidiol; and tetrahydrocannabinol (THC) and cannabidiol, or antimitotic drugs and anesthetic drugs, such as prilocaine and mitomycin; lidocaine and adriamycin; and novocaine and diclofenac.

Optionally, the active agents include a pH buffering agent. Exemplary pH buffering agents include, but are not limited to, carbonate salt, tromethamine, phosphate salts (e.g., monopotassium phosphate), citric acid, acetic acid, and acetate salt (e.g., sodium acetate). Optionally, a buffering agent included in the formulation can facilitate disintegration, erosion, and/or degradation of the mass in urine.

c. Concentrations of Active Agents

Typically, the total concentration of the one or more active agents in the formulation is typically in a range from 0.005 wt % to 20 wt %, from 0.005 wt % to 15 wt %, from 0.005% to 10% w/w, from 0.005% to 5% w/w, from 0.005% to 1% w/w, from 0.005% to 0.5% w/w, from 0.005% to 0.1% w/w, from 0.005% to 0.05% w/w, from 0.01% to 20% w/w, from 0.01% to 15% w/w, from 0.01% to 10% w/w, from 0.01% to 5% w/w, from 0.01% to 1% w/w, from 0.05% to 20% w/w, from 0.05% to 15% w/w, from 0.05% to 10% w/w, from 0.05% to 5% w/w, from 0.05% to 1% w/w, from 0.1% to 20% w/w, from 0.1% to 15% w/w, from 0.1 wt % to 10 wt %, or from 0.1 to 5 wt % of the formulation. The term “total concentration of the one or more agents” refers to the total weight of the one or more active agents relative to the total weight of the formulation. The total concentration of the one or more active agents and the concentration of each of the one or more active agents required will vary from treatment to treatment and/or subject to subject according to their need.

When the formulation contains two or more active agents, the concentration of each active agent can be in a suitable range to provide a total concentration of the one or more agents of 0.005 wt % to 20 wt %, from 0.005 wt % to 15 wt %, from 0.005% to 10% w/w, from 0.005% to 5% w/w, from 0.005% to 1% w/w, from 0.005% to 0.5% w/w, from 0.005% to 0.1% w/w, from 0.005% to 0.05% w/w, from 0.01% to 20% w/w, from 0.01% to 15% w/w, from 0.01% to 10% w/w, from 0.01% to 5% w/w, from 0.01% to 1% w/w, from 0.05% to 20% w/w, from 0.05% to 15% w/w, from 0.05% to 10% w/w, from 0.05% to 5% w/w, from 0.05% to 1% w/w, from 0.1% to 20% w/w, from 0.1% to 15% w/w, from 0.1 wt % to 10 wt %, or from 0.1 to 5 wt % of the formulation. For example, the concentration of a first active agent can be in the range of 0.001% to 19.999% w/w, 0.001% to 19% w/w, 0.001% to 15% w/w, 0.001% to 10% w/w, 0.001% to 1% w/w, 0.005% to 5% w/w, 0.005% to 2% w/w, 0.005% to 1% w/w, 0.005% to 0.5% w/w, 1% to 19% w/w, 1% to 15% w/w, 1% to 9% w/w, 1% to 8% w/w, 1% to 5% w/w, 2% to 18% w/w, from 2% to 15% w/w, from 2% to 10% w/w, from 2% to 8% w/w, or from 2% to 7% w/w, from 5% to 15% w/w, or from 5% to 10% w/w of the formulation; and the concentration of the second active agent can be 0.001% to 19.999% w/w, 0.001% to 19% w/w, 0.001% to 15% w/w, 0.001% to 10% w/w, 0.001% to 1% w/w, 0.005% to 5% w/w, 0.005% to 2% w/w, 0.005% to 1% w/w, 0.005% to 0.5% w/w, 1% to 19% w/w, 1% to 15% w/w, 1% to 9% w/w, 1% to 8% w/w, 1% to 5% w/w, 2% to 18% w/w, from 2% to 15% w/w, from 2% to 10% w/w, from 2% to 8% w/w, or from 2% to 7% w/w, from 5% to 15% w/w, or from 5% to 10% w/w of the formulation.

D. Additives

The formulation can include one or more additives. The one or more additives may be any suitable pharmaceutically acceptable excipients.

Optionally, the one or more additives include one or more molecules, oligomers, and/or polymers that are dispersible in water and alcohol.

These additives can contribute to the stability of the mass and/or the disintegration/erosion of the mass formed in urine.

1. Pharmaceutically Acceptable Excipients

Any suitable ingredients that can facilitate the handling, stability, wettability, and/or release kinetics, and/or ingredients that are needed in the manufacture process or during administration can be used as an additive. Optionally, one or more pharmaceutically acceptable excipients may be included in the formulation to facilitate disintegration, erosion, and/or degradation of the mass. For example, the formulation includes one or more disintegrants described below to facilitate disintegration, erosion, and/or degradation of the mass in urine. Optionally, one or more pharmaceutically acceptable excipients may be included in the formulation to modify or control the release of the one or more active agents from the mass, optionally to increase the time period (i.e. prolong) of the release of the one or more active agents.

Exemplary pharmaceutically acceptable excipients include, but are not limited to, plasticizers, viscosity modifiers, surfactants, permeation enhancers, diluents, lubricants, preservatives, antioxidants, disintegrants, stabilizers, or a combination thereof.

a. Surfactants

For example, the addition of one or more surfactants can modulate the disintegration of the mass in urine and the release profile of the one or more active agents. The one or more surfactants are soluble in the one or more alcohols, such as ethanol, a glycol, or a combination of ethanol and a glycol. For example, in some embodiments, the one or more surfactants are soluble in ethanol.

The surfactant can have a hydrophile-lipophile balance (HLB) from 7 to 10, such as Span® 20, Span® 80, and lecithin, which modulate the release behavior. Optionally, the surfactant has a HLB higher than 10, such as Tween® 80, Tween® 20, esters (e.g., Myrj™), and ethers (e.g., Brij®), Pluronics, Pluronic F127 which may contribute to the erosion or disintegration of the mass in urine. Optionally, the surfactant has a HLB from 1.5 to 6, such as sorbitan trioleate.

Exemplary surfactants include, but are not limited to, sorbitan fatty acids esters, such as sorbitan trioleate (HLB 1.8), sorbitan monoleate (HLB 4.3), sorbitan monolaurate (HLB 8.6); polyoxyethylene alkyl ethers, such as polyoxyethylene (2) stearyl ether (HLB 4.9), polyoxyethylene (2) cetyl ether (HLB 5.3), polyoxyethylene (4) lauryl ether (HLB 9.5), polyoxyethylene (20) stearyl ether (HLB 15.3), polyoxyethylene (21) stearyl ether (HLB 15.5), polyoxyethylene (20) cetyl ether (HLB 16.0), and polyoxyethylene (23) lauryl ether (HLB 16.0); polyoxyethylene sorbitan fatty acid esters, such as polyoxyethylene 4 sorbitan monostearate (HLB 9.6), polyoxyethylene 4 sorbitan monolaurate (HLB 9.8), polyoxyethylene 5 sorbitan monooleate (HLB 10), polyoxyethylene 20 sorbitan tristearate (HLB 10.5), polyoxyethylene 20 sorbitan trioleate (HLB 11), polyoxyethylene 20 sorbitan monoisostearate (HLB 14.9), polyoxyethylene 20 sorbitan monostearate (HLB 14.9), polyoxyethylene 20 sorbitan monooleate (HLB 15), polyoxyethylene 20 sorbitan monopalmitate (HLB 15.6), and polyoxyethylene 20 sorbitan monolaurate (HLB 16.7); polyoxyethylene stearates with HLB from 9.7 to 18.8; and poloxomers, such as poloxomer F127 (HLB 18-23).

Other exemplary surfactants include lecithin and phospholipids, and a combination thereof. Exemplary phospholipids include, but are not limited to, unsaturated phospholipids, saturated phospholipids, soy phospholipid, egg phospholipid, phosphatidylcholine (e.g. Phospholipon®, such as Phospholipon® 90G and Phospholipon® 90H, and Lipoid®, such as Lipoid® 100 and Lipoid® 75), and a combination thereof.

b. Other Exemplary Pharmaceutically Acceptable Excipients

Examples of suitable additives include, but are not limited to, polyethylene glycol, triacetin, dimethyl phthalate, diethyl phthalate, dibutyl phthalate, dibutyl sebacate, triethyl citrate, tributyl citrate, triethyl acetyl citrate, and acetylated monoglycerides.

Examples of suitable preservatives include, but are not limited to, benzyl alcohol, phenol, phenoxyethanol, benzoic acid, alkyl esters of p-hydroxy benzoic acid such as methyl, ethyl, propyl, butyl and heptyl (collectively known as “parabens”), or a combination thereof.

Disintegrants can be included, particularly to facilitate the disintegration or erosion of the capture mass in urine. Suitable disintegrants include, but are not limited to, any surfactants as described above, hydroxypropyl cellulose, clays, gums, and cross linked polymers, such as polyvinylpyrrolidone (PVP).

2. Water- and Alcohol-Dispersible Molecules/Oligomers/Polymers

Typically, molecules, oligomers, or polymers that are dispersible in water and in alcohol(s), such as ethanol or propylene glycol, or a combination thereof, can be included in the formulations.

The formulations can include one or more water- and alcohol-dispersible molecules, water- and alcohol-dispersible oligomers, or water- and alcohol-dispersible polymers, or a combination thereof to modulate the release properties, by modulating the disintegration/erosion and/or the stability of the mass in urine. Generally, “oligomer” refers to a molecule that contain a few repeating units, such as two, three, four, five, six, seven, eight, nine, or ten repeating units, while “polymer” refers to a molecule that contain more than ten repeating units.

Exemplary polymers that are water- and alcohol-dispersible include, but are not limited to, Klucel™ (hydroxypropylcellulose), Carbopol® (polyacrylic acid polymer), polyvinyl acetate phthalate (PVAP), polyvinylpyrrolidone (PVP), poloxamers such as poloxamer 407, and Pluronic®, or a combination thereof.

3. Concentrations of Additives

Typically, the total concentration of the one or more additives is in a range from 0.1 wt % to 20 wt %, from, from 0.1 wt % to 15 wt %, from 1 wt % to 12 wt %, from 1 wt % to 10 wt %, from 1 wt % and 15 wt %, from 2 wt % to 20 wt %, from 2 wt % to 15 wt %, from 2 wt % to 10 wt %, from 3 wt % to 20 wt %, from 3 wt % to 15 wt %, or from 3 wt % to 10 wt % of the formulation. The term “total concentration of the one or more additives” refers to the total weight of the one or more additives relative to the total weight of the formulation.

Optionally, the formulation includes one or more additives that contain one or more surfactants with an HLB from 7 to 10 and the total concentration of the one or more surfactants is in a range up to 20% w/w, up to 15% w/w, up to 8% w/w, from 0.1% to 10% w/w, or from 2% to 5% w/w of the formulation. In some embodiments, the formulation includes one or more additives that include sorbitan laurate (e.g. Span® 20), optionally the total concentration of the sorbitan laurate is in a range up to 20% w/w, 15% w/w, up to 8% w/w, from 0.1% to 10% w/w, from 1% to 5% w/w, or from 2% to 6% w/w of the formulation. In some embodiments, the formulation includes one or more additives that include soy phospholipid or egg lecithin, optionally the concentration of the soy phospholipid or egg lecithin is in a range up to 20% w/w, 15% w/w, up to 8% w/w, from 0.1% to 10% w/w, from 1% to 7% w/w, or from 2% to 6% w/w of the formulation.

Optionally, the one or more additives contain one or more water- and alcohol-dispersible molecules described above, and the total concentration of the one or more water- and alcohol-dispersible molecules is in a range up to 20% w/w, 15% w/w, up to 8% w/w, from 0.1% to 10% w/w, from 2% to 4% w/w, from 1% to 5% w/w, or from 2% to 6% w/w of the formulation.

Optionally, the formulation includes one or more additives that contain one or more water- and alcohol-dispersible polymers described above and the total concentration of the one or more water- and alcohol-dispersible polymers is in a range up to 8% w/w, from 0.2% to 5% w/w, from 1% to 6% w/w, from 0.1% to 4% w/w of the formulation.

Optionally, the formulation includes one or more additives that include a hydroxypropylcellulose (e.g., Klucel™ HF, Klucel™ EF, or a combination thereof) and the concentration of the hydroxypropylcellulose is up to 8% w/w, from 0.2% to 5% w/w, from 1% to 6% w/w, from 0.1% to 4% w/w of the formulation.

4. Exemplary Formulations

An exemplary formulation includes an ethylcellulose, a polymethacrylate, or a combination of ethylcellulose and Eudragit®; any one or more active agents as described above; and optionally any one or more additives as described above, in a suitable solvent, such as ethanol, propylene glycol, or a combination of ethanol and propylene glycol, optionally containing water in a concentration of up to 20% w/w of the formulation.

a. Exemplary Formulations containing Lidocaine

Some formulations contain lidocaine. The lidocaine present in the formulations can have a concentration in a range from 1% to 5% w/w of the formulation. The lidocaine present in the formulations may be in the base form, in a pharmaceutical salt form (e.g., lidocaine HCl), or a combination of the base form and a pharmaceutical salt form (e.g., lidocaine base and lidocaine HCl).

For these formulations, the polymers in the composition can include a Eudragit® polymer (e.g., Eudragit® S100, Eudragit® L100, Eudragit® E100, Eudragit® RS100, Eudragit® RL100, Eudragit® ES100, or Eudragit® EL100) or a combination of Eudragit® of different types; an ethylcellulose; or a combination of a Eudragit® polymer and an ethylcellulose polymer. For formulations containing a Eudragit® polymer, the Eudragit® polymer is present at a concentration from 3% to 8% w/w of the formulation. For formulations containing ethylcellulose, the ethylcellulose is present at a concentration from 3% to 6% w/w of the formulation.

Optionally, these formulations also contain an additive, such as Span® 20 and/or a phospholipid. Each of the additives can be present in a concentration from 2% to 12% w/w of the formulation.

For these lidocaine formulations, the solvent can be ethanol, propylene glycol, or a combination of ethanol and propylene glycol. The ethanol present in these formulations can have a concentration from 40% to 90% w/w of the formulation. The propylene glycol present in these formulations can have a concentration from 35% to 50% w/w of the formulation.

Optionally, exemplary formulations contain lidocaine in combination with another active agent or additive, such as cannabidiol, oxybutynin, or tromethamine. In these formulations, the additional active agent can be present in a concentration from 0.1% to 4% w/w of the formulation.

b. Exemplary Formulations Containing Cannabidiol

Some formulations contain cannabidiol (CBD). The cannabidiol present in the formulations can have a concentration in a range from 0.1% to 0.5% w/w of the formulation.

For these formulations, the polymers in the composition can include a Eudragit® polymer (e.g., Eudragit® S100, Eudragit® E100, or Eudragit® ES100) or a combination of Eudragit® of different types; an ethylcellulose; or a combination of a Eudragit® polymer and an ethylcellulose polymer. For formulations containing a Eudragit® polymer, the Eudragit® polymer is present at a concentration from 5% to 10% w/w of the formulation. For formulations containing ethylcellulose, the ethylcellulose is present at a concentration from 2% to 7% w/w of the formulation. Optionally, these formulations also contain an additive, such as a surfactant (e.g. Span®20, HPC, and/or Tween 80) and/or a water- and alcohol-dispersible polymer. Each of the additives can be present in a concentration from 2% to 11% w/w of the formulation.

For these cannabidiol formulations, the solvent can be ethanol, propylene glycol, or a combination of ethanol and propylene glycol. The ethanol present in these formulations can have a concentration from 50% to 90% w/w of the formulation. The propylene glycol present in these formulations can have a concentration from 25% to 40% w/w of the formulation.

Optionally, exemplary formulations may contain cannabidiol in combination with another active agent, such as oxybutynin, gentamycin, or lidocaine. In these formulations, the additional active agent can be present in a concentration from 0.5% to 2.5% w/w of the formulation.

c. Exemplary Formulations containing Ibuprofen

Some formulations contain ibuprofen. The ibuprofen present in the formulations can have a concentration in a range from 5% to 10% w/w of the formulation.

For these formulations, the polymers in the composition can include a Eudragit® polymer (e.g., Eudragit® S100 or Eudragit® E100) or a combination of Eudragit® of different types; an ethylcellulose; or a combination of a Eudragit® polymer and an ethylcellulose polymer. For formulations containing a Eudragit® polymer, the Eudragit® polymer is present at a concentration from 4% to 7% w/w of the formulation. For formulations containing ethylcellulose, the ethylcellulose is present at a concentration from 4% to 5% w/w of the formulation.

Optionally, these formulations also contain an additive, such as Span® 20, HPC, a buffering agent, a surfactant such as polysorbate 20 or polysorbate 80, PLGA, and/or PVA. Each of the additives can be present at a concentration from 2% to 5% w/w of the formulation.

For these ibuprofen formulations, the solvent can be ethanol, propylene glycol, or a combination of ethanol and propylene glycol. The ethanol present in these formulations can have a concentration from 10% to 49% w/w of the formulation. The propylene glycol present in these formulations can have a concentration from 41% to 95% w/w of the formulation.

d. Exemplary Formulations Containing Oxybutynin

Some formulations contain oxybutynin. The oxybutynin present in the formulations can have a concentration in a range from 0.1% to 4% w/w of the formulation. The oxybutynin present in the formulations may be in the base form, in a pharmaceutical salt form (e.g., oxybutynin HCl), or a combination of the base form and a pharmaceutical salt form (e.g., oxybutynin base and oxybutynin HCl).

For these formulations, the polymers in the composition can include a Eudragit® polymer (e.g., Eudragit® S100, Eudragit® RS100, or Eudragit® EL100) or a combination of Eudragit® of different types; an ethylcellulose; or a combination of a Eudragit® polymer and an ethylcellulose polymer. For formulations containing a Eudragit® polymer, the Eudragit® polymer is present at a concentration from 2% to 9% w/w of the formulation. For formulations containing ethylcellulose, the ethylcellulose is present at a concentration from 3% to 5% w/w of the formulation.

Optionally, these formulations also contain an additive, such as Span® 20, a phospholipid, a surfactant, HPC, a buffering agent, carbonate salt, phosphate salt, tromethamine and/or benzyl alcohol. Each of the additives can be present in a concentration from 0.5% to 10% w/w of the formulation.

For these oxybutynin formulations, the solvent can be ethanol, propylene glycol, or a combination of ethanol and propylene glycol. The ethanol present in these formulations can have a concentration from 20% to 92% w/w of the formulation. The propylene glycol present in these formulations can have a concentration from 20% to 87% w/w of the formulation.

Optionally, exemplary formulations may contain oxybutynin in combination with another active agent, such as vitamin E or lidocaine HCl. In these formulations, the additional active agent can be present in a concentration from 0.4% to 3% w/w of the formulation.

e. Exemplary Formulations Containing Doxorubicin

Some formulations contain doxorubicin. The doxorubicin present in the formulations can have a concentration in a range from 0.1% to 0.3% w/w of the formulation.

For these formulations, the polymers in the composition can include a Eudragit® polymer (e.g., Eudragit® RS100, or Eudragit® L100) or a combination of Eudragit® of different types. For formulations containing a Eudragit® polymer, the Eudragit® polymer is present at a concentration from 5% to 8% w/w of the formulation.

Optionally, these formulations also contain an additive, such as sorbitan ester, Span® 20, HPC, and/or a buffering agent. Each of the additives can be present at a concentration from 2% to 7% w/w of the formulation.

For these doxorubicin formulations, the solvent can be ethanol, propylene glycol, or a combination of ethanol and propylene glycol. The ethanol present in these formulations can have a concentration up to 90% w/w, from 10% to 50% w/w, or from 50% to 90% w/w of the formulation. The propylene glycol present in these formulations can have a concentration up to 40%, from 35% to 37% w/w, or from 30% to 90% w/w of the formulation.

f. Exemplary Formulations Containing Tramadol

Some formulations contain tramadol. The tramadol present in the formulations can have a concentration in a range from 1% to 10% w/w of the formulation. The tramadol present in the formulations may be in the base form, in a pharmaceutical salt form (e.g., tramadol HCl), or a combination of the base form and a pharmaceutical salt form (e.g., tramadol base and tramadol HCl).

For these formulations, the polymers in the composition can include a Eudragit® polymer (e.g., Eudragit® E100, Eudragit® S100, Eudragit® ES100, Eudragit® RS100, or Eudragit® RL100) or a combination of Eudragit® of different types. For formulations containing a Eudragit® polymer, the Eudragit® polymer is present at a concentration from 2% to 8% w/w of the formulation.

Optionally, these formulations also contain an additive, such as Span® 20, a surfactant such as polysorbate 20 or polysorbate 80, HPC (e.g. Klucel™), a buffering agent, and/or a preservative such as benzyl alcohol. Each of the additives can be present at a concentration from 1% to 12% w/w of the formulation.

For these tramadol formulations, the solvent can be ethanol, propylene glycol, or a combination of ethanol and propylene glycol. The ethanol present in these formulations can have a concentration from 40% to 65% w/w of the formulation. The propylene glycol present in these formulations can have a concentration from 20% to 55% w/w of the formulation.

g. Exemplary Formulations Containing Mytomycin C

Some formulations contain mytomycin C. The mytomycin C present in the formulations can have a concentration in a range from 0.2% to 0.5% w/w of the formulation.

For these formulations, the polymers in the composition can include a Eudragit® polymer (e.g., Eudragit® ES100) or a combination of Eudragit® of different types; an ethylcellulose; or a combination of a Eudragit® polymer and an ethylcellulose polymer. For formulations containing a Eudragit® polymer, the Eudragit® polymer is present at a concentration from 6% to 8% w/w of the formulation. For formulations containing ethylcellulose, the ethylcellulose is present at a concentration about 1% w/w of the formulation

Optionally, these formulations can also contain an additive, such as Span®20, a phospholipid, HPC, and/or a buffering agent. Each of the additives can be present at a concentration from 2% to 8% w/w of the formulation.

For these Mytomycin C formulations, the solvent can be ethanol, propylene glycol, or a combination of ethanol and propylene glycol. The ethanol present in these formulations can have a concentration from 30% to 65% w/w of the formulation. The propylene glycol present in these formulations can have a concentration from 25% to 55% w/w of the formulation.

h. Exemplary Formulations Containing Mirabegron

Some formulations contain mirabegron. The mirabegron present in the formulations can have a concentration in a range from 0.05% to 0.2% w/w of the formulation.

For these formulations, the polymers in the composition can include a Eudragit® polymer (e.g., Eudragit® S100 or Eudragit® ES100) or a combination of Eudragit® of different types. For formulations containing a Eudragit® polymer, the Eudragit® polymer is present at a concentration from 5% to 8% w/w of the formulation.

Optionally, these formulations can also contain an additive, such as Span® 20. Each of the additives can be present at a concentration from 2% to 10% w/w of the formulation.

For these mirabegron formulations, the solvent can be ethanol, propylene glycol, or a combination of ethanol and propylene glycol. The ethanol present in these formulations can have a concentration from 45% to 50% w/w of the formulation. The propylene glycol present in these formulations can have a concentration from 30% to 45% w/w of the formulation.

i. Additional Exemplary Formulations

Additional exemplary formulations contain hydrocortisone, bupivacaine, naproxen, triamcinolone, thiotepa, gemcitabine, ciprofloxacin, diclofenac sodium, adriamycin, erytomycin ethylsuccinate, or fentamyl, or combinations thereof.

For formulations containing any of these drugs, the polymers in the composition can include a Eudragit® polymer (e.g., Eudragit® S100, Eudragit® RS100, Eudragit® ES100, or Eudragit® EL100) or a combination of Eudragit® of different types; an ethylcellulose; or a combination of a Eudragit® polymer and an ethylcellulose polymer.

Optionally, formulations containing any of these drugs also contain an additive, such as Span®20, a phospholipid, carbopol, Klucel™, lecithin, HPC, polycarbophil, a surfactant, and/or a plasticizer such as triacetin, and/or a buffering agent such phosphate, carbonate or tromethamine.

For formulations containing any of these drugs, the solvent can be ethanol, propylene glycol, or a combination of ethanol and propylene glycol.

More specific exemplary formulations are described in the Examples.

C. Mass

A mass is formed instantaneously in urine (i.e., within 5 second upon formulation contact with urine) by precipitation of the one or more polymers when the formulation is in contact with urine. Human urine pH can range from about 4.5 to about 8. The mass entraps the one or more active agents and can release the active agents over an extended period of time. The term “extended period” generally refers to a time period over 30 minutes, such as at least 1 hour, 2 hours, 4 hours, 8 hours, 12 hours, 16 hours, 24 hours, 28 hours, 32, hours 36 hours, 40 hours, 44 hours, 48 hours, or up to 1 month after instillation.

Prolonged release of an active agent from a mass can be evaluated using a suitable model, such as the in vitro bladder simulation study by Sang Hyun Kim et al, The Korean Journal of Urological Oncology, 15(3):178-186 (2017) or the ex vivo method presented by Gross et al., Journal of Urology, 183(4):e74-e75 (2010) or a modification thereof, such as the method described in Examples 76 and 78.

A prolonged release profile for active agent(s) from a capture mass formed in contact with urine can be demonstrated using standard in vivo animal studies, such as in vivo studies in swine described in Example 81. For example, tested urine samples of an animal receiving the formulation can show the presence of the active agent(s) in the urine samples over an extended period of time, even when subjected to voiding. These results can be compared to a control containing the same active agent(s) at the same concentration as the test formulation in the same solvent (i.e. the same alcohol or miscible alcohols), but in the absence of any polymers or additional additives. The tested urine samples of an animal receiving the control can show a decrease of the concentration of the active agent(s) in the urine sample over the same period of time due to urination.

Typically, the active agent(s) in the control has the same weight and the same concentration (w/w) as that in the formulation. For example, in the formulation, the active agent is 20 mg and the concentration of the active agent(s) is 1% w/w of the formulation that includes the solvent, the polymer(s), and the active agent(s); in the control, the active agent is also 20 mg and the concentration of the active agent(s) is also 1% w/w of the control that includes the solvent and the active agent(s). If any additives are included in the formulation, typically such additives would not be included in the control.

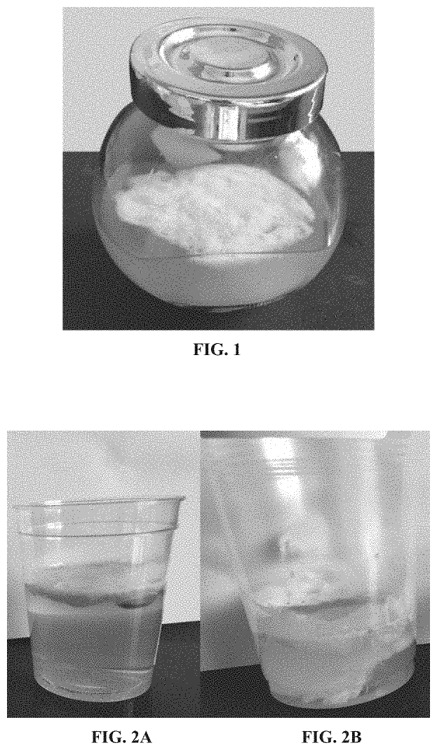

An exemplary mass is depicted in FIG. 1. As shown in FIG. 1, the mass 10 is formed in urine 20 by precipitation of polymers that entraps the one or more active agents. The mass 10 can float on the surface of urine 20. The mass 10 depicted in FIG. 1 has a largest dimension of about 6 cm.

1. Size

Typically, the in situ formed mass in urine has a largest dimension in a range from 0.5 cm to 20 cm, from 8 mm to 12 cm, or from 2 cm to 7 cm, such as from about 4 cm to about 6 cm. The term “largest dimension” refers to the longest distance between two edges forming the borders of the mass.

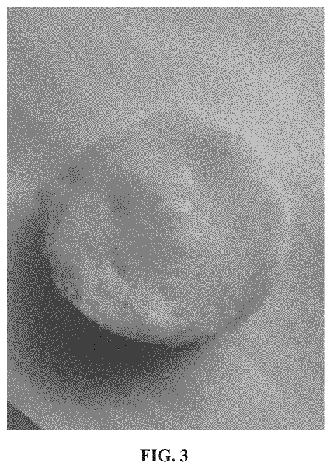

Measurement of the largest dimension (d) of an exemplary mass 10′″ (removed from urine after its formation) is illustrated in FIG. 3. As shown in FIG. 3, the largest dimension (d) is the longest distance between the two edges 12 and 14 of the mass 10′″.

The dimensions of the mass can be modulated mainly by the instilled formulation volume, and/or by the polymer(s), the additive(s), the concentrations of the polymer(s) and/or additive(s).

2. Location

The in situ formed mass can float on the surface of the urine or be immersed in the urine. FIG. 2A shows an exemplary mass 10′ formed in urine that floats on the surface of the urine. FIG. 2B shows an exemplary mass 10″ formed in urine that is immersed in the bulk of the urine.

3. Release Properties

The capture mass entraps the one or more active agents and provides prolonged release of the active agent. The capture mass releases the active agents over an extended period of time, for example, up to 1 hour, up to 2 hours, up to 5 hours, up to 8 hours, up to 24 hours, up to 48 hours, up to 3 days, up to 4 days, up to 5 days, up to 6 days, up to 7 days, up to 15 days, up to 30 days, or more than 30 days. The release of the active agents can start immediately upon mass formation and continue over an extended period of time.

Release of active agents from the mass can occur via disintegration and/or erosion of the mass, and/or diffusion of the active agents over a period of time, for example, up to 1 hour, up to 2 hours, up to 5 hours, up to 8 hours, up to 24 hours, up to 48 hours, up to 3 days, up to 4 days, up to 5 days, up to 6 days, up to 7 days, up to 15 days, up to 30 days, or more than 30 days. The release of active agents from the mass can be demonstrated using an in vitro test such as described in Examples 78 and 80. For example, as described in Example 74, where methylene blue entrapped in a mass formed in simulated urine fluid (SUF) was released into the urine over a period of 48 hours, where the color of the urine was observed at various time points ranging from 3 hours to 48 hours. Each urine sample at each time point (i.e. from 3 to 48 hours) was blue, demonstrating that the formulations described herein are able to provide prolonged release of active agents.

Prolonged release of entrapped tramadol was also demonstrated in Example 78, where tramadol was released for at least 22 hours following injection of the formulation into simulated urine fluid (SUF) and formation of a capture mass in the SUF. Prolonged release of entrapped tramadol was also demonstrated in Example 80, where tramadol was released for at least 24 hours following injection of formulations 1 and 2 and for at least 16 hours following injection of formulation 3 into simulated urine fluid (SUF) and formation of a capture mass in the SUF.

The release properties of the mass can be modulated by the polymer(s), the additive(s), the active(s), the concentrations of the polymer(s), additive(s), and/or active agent(s), and/or the pH of the urine in the urinary tract, the bladder, or the kidney(s). For example, when the composition contains both hydrophobic active agents and hydrophilic active agents, the hydrophilic form is released more quickly, while the hydrophobic form has a prolonged release and is released in the urine over an extended period of time. This results in a drug release profile with a quick onset and a prolonged release.

When the mass contains one or more polymers that are pH-dependent (i.e. solubility of the polymer in urine changes with a change of urine pH, such as Eudragit® S100 and Eudragit® L100), adjusting the pH of urine can modulate the release profile of the one or more active agents from the mass. For example, for a mass that contains Eudragit® L100, a more basic urine, such as with a pH in the range of about 6.5 to about 8, can lead to faster degradation of the mass compared to the same mass in urine having a lower pH, such as a pH lower than 6.5, optionally in the range of about 4.5 to about 6.5. Thus, in urine with a basic pH, the mass releases the one or more active agents more quickly into the urine mass compared to release from the same mass in urine having a pH lower than 6.5 such as in the range of about 4.5 to about 6.5.

II. Pre-Filled Syringe or Container Containing the Formulation or a Pre-Formulation (i.e. the Components of the Formulation without the Active Agent(s))

a. Pre-Filled Container or Syringe Containing a Formulation

The formulations can be provided in a pre-filled syringe or container. Typically, the container or syringe and the formulation therein are sterile. The formulation can be is sterilized by a method described below.

Optionally, the formulation is stored in a syringe. A pre-filled syringe containing the formulation can be provided. At the time of use, the user can connect the pre-filled syringe to a catheter, a cystoscope, or an ureteroscope inserted into the urinary tract, the bladder, an/or the kidney(s) of a patient and then depress the syringe to instill the formulation into the patient's urinary tract, the bladder, an/or the kidney(s).

Optionally, the formulation is stored in a pharmaceutically acceptable container, such as a vial. The vial containing the formulation can be provided to the user prior to instillation such that the user can fill the formulation in a syringe and connect the syringe to a catheter, a cystoscope, or an ureteroscope inserted into the urinary tract, the bladder, an/or the kidney(s) of a patient to start instillation.

b. Pre-Filled Container or Syringe Containing a Pre-Formulation

The dry form of many drugs is more stable and can be stored for a longer time period than the liquid form. In some embodiments, the one or more active agents to be instilled in a patient are provided in a dry form separately from the other components that form the final formulation. A pre-formulation refers to the ingredients in the form of a liquid to which the one or more active agents will be added to form the formulation to be instilled in a patient. For example, the pre-formulation contains at least the one or more alcohols and the one or more polymers, optionally with one or more additives, as described above.

The pre-formulation may be provided in a container or syringe.

The pre-formulation and dry active agent(s) can be provided in separate containers in a kit. The kit can contain a first container that is pre-filled with the liquid pre-formulation and a second container that contains the one or more active agents in a solid form. Optionally, if two or more active agents are provided, each of the active agents can be in solid form and can be stored in a separate container. Alternatively, one or more active agents can be stored in one compartment of a container and one or more different active agents can be stored in a separate compartment of the same container.

A kit containing a first container pre-filled with the liquid pre-formulation and a second container containing the solid active agent(s) can be provided to a user such that the user can mix the active agent(s) with the pre-formulation prior to instillation.

The kit may also contain instructions for combining the pre-formulation and the active agent(s) and/or a device for mixing the pre-formulation and the active agent(s). The kit may also contain a pharmaceutically acceptable device for instillation, such as a syringe.

When the active agent(s) is provided as a dry powder, the kit may contain an additional alcoholic solvent and instructions for preparing active agent solution of the active agent(s) prior to mixing with the pre-formulation. The additional alcoholic solvent may be provided in a separate container included in the kit. Alternatively, the dry active agent(s) may be mixed with the pre-formulation by directly adding the pre-formulation into the container containing the active agent(s).

Optionally, the kit contains a dual-chamber, needle-free device or a dual-chamber syringe. When a dual chamber device or syringe is provided, the pre-formulation can be stored in the back chamber and the one or more active agents can be stored in the front chamber. Alternatively, the pre-formulation can be stored in the front chamber and the one or more active agents can be stored in the back chamber. When the active agent(s) is stored as a dry powder in the kit, the pre-formulation is typically stored in the back chamber and the dry active agent(s) is stored in the front chamber. When a dual-chamber, needle-free device is provided, the kit may contain a needle to be attached to the device prior to instillation.

Typically, the formulation or pre-formulation and the active agent(s) are provided in sterile form. Typically, the container or syringe containing the formulation or pre-formulation and the active agent(s) is sterile.

Optionally, the pharmaceutically acceptable device and/or containers are sterilized prior to filling the containers, compartments thereof, or device with the formulation or the pre-formulation and the active agent(s) using a suitable sterilization method. Suitable methods for sterilizing the formulation, the pre-formulation, the containers, compartments, and/or syringe include, but are not limited to, gas sterilization, irradiation sterilization, heat sterilization, filtration, and aseptic filling, or a combination thereof.

### III. Methods of Using the Formulations

A. Treating or Ameliorating Symptoms Associated with Disorders/Diseases in or Affecting Urinary Tract, Bladders and/or Kidneys

Methods of using the formulations for treating or ameliorating one or more symptoms associated with a disorder or disease in or affecting a patient's urinary tract, bladder, and/or kidney(s) are disclosed.

Generally, the method includes (i) inserting a catheter, a cystoscope, or an ureteroscope into the urinary tract, the bladder, and/or the kidney(s) of the patient, and (ii) instilling the formulation into the urinary tract, the bladder, and/or the kidney(s) through the catheter, the cystoscope, or the ureteroscope. Following step (ii), the instilled formulation forms a mass in the patient's urine and the one or more active agents in the formulation are entrapped in the mass.

1. Inserting a Catheter, a Cystoscope, or an Ureteroscope into Urinary tract, Bladder, and/or Kidney(s)

Generally, prior to instillation, a hollow device for delivering the formulation is inserted into the urinary tract, the bladder, and/or the kidney(s) of the patient. Exemplary suitable devices for instillation include a urinary catheter, a cystoscope, and an ureteroscope.

Procedures of inserting a device for instillation into the urinary tract, the bladder, and/or the kidney(s) of a patient are known. For example, when using a cystoscope in the method, the cystoscope is advanced via the urethra into the urinary bladder. A prefilled syringe with the formulation is connected to the cystoscope working channel via a Luer lock connector. The formulation is then instilled to the bladder by pushing the syringe plunger. The cystoscope is subsequently removed. 2. Instilling the Formulation into Urinary Tract, Bladder and/or Kidney(s)

Generally, the user instills the formulation into the urinary tract, the bladder, and/or the kidney(s) of the patient through the catheter, the cystoscope, or the ureteroscope. The user can be the medical professional or the patient being treated (e.g. self-instillation). The instillation process typically involves attaching a syringe to the end of a catheter, a cytoscope, or an ureteroscope and depressing the syringe in a swift action, similar to administering an injection to a patient.

Optionally, the user, uses a pre-filled syringe containing the formulation for instillation and attaches the pre-filled syringe to syringe to the end of a catheter, a cytoscope, or an ureteroscope prior to instillation.

Embodiments in which the formulation is formed, such as by mixing one or more active agents with a liquid pre-formulation, prior to instillation are described below.

Immediately following instillation, the formulation forms a mass in the patient's urine.

a. Forming a Mass

The formulation is instilled into the urinary tract, the bladder, and/or the kidney(s) and contacts the urine inside the urinary tract, the bladder, and/or the kidney(s). Instantaneously upon contact, the one or more polymers in the formulation precipitate from the formulation and entraps the one or more active agents to form a mass in the patient's urine. Optionally, the one or more polymers in the formulation precipitate from the formulation and agglomerate to form a mass that entraps the one or more active agents in the patient's urine. For example, the formulation forms a mass in the patient's urine within 1 second upon contact with the urine. The mass entraps the one or more active agents in the formulation.

The mass typically has a largest dimension in a range from 0.5 cm to 20 cm, from 8 mm to 12 cm, or from 2 cm to 7 cm, such as from about 4 cm to about 6 cm. For example, the mass can have a largest dimension of about 6 cm. The dimensions of the mass can be modulated by the instilled formulation volume, the polymer(s), the additive(s), the concentrations of the polymer(s) and/or additive(s), and/or the rate of instillation in the urinary tract, the bladder, or the kidney(s).

The mass can float on the surface of the urine or be immersed in the urine, see, for example, FIGS. 2A and 2B.

i. Mass Entrapping Active Agents

The one or more active agents entrapped in the mass may be present inside the mass, on the surface of the mass, and/or dispersed throughout the mass Typically, the active agents present inside the mass are released over a period of time.

ii. Releasing Active Agents

The mass can remain in the urinary tract, the bladder and/or the kidney(s) without obstructing the urethra, thereby release the entrapped active agents into the urine for an extended period of time, for example, up to 1 hour, up to 2 hours, up to 5 hours, up to 8 hours, up to 24 hours, up to 48 hours, up to 3 days, up to 4 days, up to 5 days, up to 6 days, up to 7 days, up to 15 days, up to 30 days, or more than 30 days. The mass releases the entrapped active agents as a result of disintegration and/or erosion of the mass, and/or diffusion of the active agents.

b. Instillation Volume

Typically, the formulation has an instillation volume from 0.5 mL to 120 mL, from 4 mL to 100 mL, from 2 mL to 60 mL, from 5 mL to 40 mL, from 1 mL to 30 mL, from 1 mL to 10 mL, or from 1 mL to 5 mL.

The instillation volume affects the dimension of the mass formed and the administrated amount of active agents, thereby affects the release profile. Typically, the larger the instillation volume is, the larger the dimension of the mass and the larger the amount of the active agents.

3. Optional Steps

In addition to the steps described above, which include: step (i) inserting a catheter, a cystoscope, or an ureteroscope into the urinary tract, the bladder, and/or the kidney(s) of the patient, and step (ii) instilling the formulation into the urinary tract, the bladder, and/or the kidney(s) through the catheter, the cystoscope, or the ureteroscope, the method may include one or more additional steps. The additional steps can occur prior to step (i) and/or subsequent to step (i) but prior to step (ii).

a. Mixing the Active Agents with a Pre-formulation not Containing Active Agents

The method may include a step of mixing the one or more active agents with the one or more polymers and the solvent prior to step (ii).

Optionally, a pre-formulation is provided where the pre-formulation contains a solvent and one or more polymers where the one or more polymers are dissolved in the solvent. Optionally, the pre-formulation also includes one or more additives as described above.

A user, such as a medical professional or the patient being treated, can mix the separately provided active agents with the pre-formulation to form the formulation prior to instillation.

Optionally, the user, can select one or more active agents according to a patient's needs and mix with the pre-formulation to form a formulation that meet the patient's needs.

b. Filling the Formulation into a Suitable Device for Instillation

The method may include a step of filling the formulation into a suitable device for instillation prior to step (ii).

Optionally, a user, such as a medical professional or the patient being treated, can fill the formulation into a syringe prior to instillation. Optionally, the user, can sterilize the formulation and/or the syringe prior to filling the formulation into the syringe.

Optionally, the formulation is pre-sterilized and stored in a vial. The user can fill the pre-sterilized formulation into a syringe for instillation. The pre-sterilized formulation can be provided in a vial as a kit as described above.

Optionally, a kit containing two or more containers where a pre-formulation and one or more active agents are stored in separate containers as described above is used to form the formulation prior to instillation. When a pre-formulation is provided, the user can mix the pre-formulation and the one or more active agents and fill the mixed formulation into a syringe prior to instillation.

Optionally, the user uses a pre-filled dual-chamber, needle-free device or a dual-chamber syringe, where a pre-formulation is stored in one chamber and one or more active agents are stored in the other chamber as described above. A suitable needle (which may be provided in the kit containing the dual-chamber, needle-free device) can be attached to the device prior to instillation.

c. Repeating Instillation of the Formulation

The method can include a step of repeating step (ii) instilling the formulation into the urinary tract, the bladder, and/or the kidney(s) through the catheter, the cystoscope, or the ureteroscope to form a second mass. Step (ii) may be repeated at least one time, at least two times, at least three times, at least five times, at least ten times, at least twenty times, up to thirty times, or more than thirty times. For example, the step (ii) is repeated one time, two times, three times, five times, ten times, fifteen times, twenty times, or thirty times.

Each repeated instillation may use the same or a different formulation from the previous instillation. For example, a second instillation uses a formulation containing different active agents and/or having a different instillation volume from the first instillation. Optionally, the second instillation may also use a formulation containing a different carrier, different polymers and/or different additives from the first instillation. The change of formulation can generate masses of similar or different dimensions in the urine.

i. Consecutive Instillations

The repeating step may be performed consecutively following the first instillation. For example, the instillation is repeated within 10 minutes, within 8 minutes, within 5 minutes, within 3 minutes, within 2 minutes, within 1 minute, or within 30 seconds following the previous instillation.

ii. Instillations at Different Times

Optionally, the repeating step is performed regularly at a different time. For example, the instillation may be performed at a frequency, such as every hour, every 2 hours, every 5 hours, every 8 hours, every day, every 2 days, every 3 days, every 5 days, every 7 days, every 10 days, every 30 days. Alternatively, the instillation may be repeated irregularly, for example, repeating the instillation 1 day after the first instillation, then 2 days after the second instillation, then 5 days after the third instillation, then 7 day after the fourth instillation, and then 30 days after the fifth instillation. The time interval between instillations are determined based on the patient's needs.

c. Adjusting Release Rate of the Active Agents

The method can include a step of adjusting release rate of the one or more active agents from the mass. The release rate may be accelerated or delayed based on patient's needs. Optionally, the adjusting step is performed prior to, during, and after any one or more of the step described above.

i. Accelerating Release

Optionally, the release of the one or more active agents can be accelerated by accelerating disintegration and/or erosion of the mass. For example, a user, such as a medical professional, can raise the pH of the urine by instilling a basic solution into the urinary tract, the bladder, and/or the kidney(s) of the patient, administering to the patient bicarbonate medications or food associated with raising the urine pH, or a combination thereof. Foods associated with raising the urine pH is known, for example, nuts, beet, almond, cauliflower, avocado, and citrus fruit. Optionally, the patient can self-administer any of these solutions, medications, or foods.

Optionally, the method includes raising the pH of the urine prior to step (i), subsequent to step (i) and prior to step (ii), or subsequent to step (ii) described above. Optionally, the step of accelerating disintegration and/or erosion of the mass can be repeated. For example, this step can be performed prior to step (i) and repeated subsequent to step (i) and prior to step (ii), and/or repeated subsequent to step (ii). For example, a patient is administered (or self-administers) a bicarbonate medication prior to step (i) and is administered the same or a different bicarbonate medication subsequent to step (i) and prior to step (ii), and optionally, is administered the same or a different bicarbonate medication again subsequent to step (ii).

ii. Delaying Release

Optionally, the release of the one or more active agents can be delayed by slowing down the disintegration and/or erosion of the mass. For example, a user, such as a medical professional, can lower the pH of the urine by administering to the patient food associated with lowering the urine pH. Foods associated with lowering the urine pH is known, for example, high protein food such as meat, fish, and poultry. Optionally, the patient can self-administer any of these foods.

Optionally, the method includes lowering the pH of the urine prior to step (i), subsequent to step (i) and prior to step (ii), or subsequent to step (ii) described above. Optionally, the step of lowering disintegration and/or erosion of the mass can be repeated. For example, this step can be performed prior to step (i) and repeated subsequent to step (i) and prior to step (ii), and/or repeated subsequent to step (ii). For example, a patient is administered food associated with lowering the urine pH prior to step (i) and is administered the same or a different food associated with lowering the urine pH subsequent to step (i) and prior to step (ii), and optionally, is administered the same or a different food associated with lowering the urine pH again subsequent to step (ii).

4. Disorders and Diseases in or Affecting Urinary Tract, Bladders and/or Kidneys

The methods described herein are suitable for treating or ameliorating one or more symptoms associated with a disorder or disease in or affecting a patient's urinary tract, bladder, and/or kidney(s), for example, cancer (i.e., carcinoma, wall superficial cancer, etc.), bladder interstitial pain, interstitial cystitis, sphincter dysfunction, infection, incontinence, over active bladder, urinary bladder dysfunction, trigonitis, inflammation, local anesthesia, chemotherapy, radioactive effect, urine pH modification. The methods are also suitable for diagnosing a disorder in or affecting a patient's urinary tract, bladder, and/or kidney(s).

Optionally, the methods are suitable for treating or ameliorating one or more symptoms associated with a disorder or disease in or affecting a patient's genitourinary tract and/or prostate ailments.

a. Urinary Tract, Bladder, and Kidney Inflammation

Optionally, the patient being treated may have one or more symptoms associated with inflammation of the urinary tract, the bladder, and/or the kidney(s), such as interstitial cystitis, radiation cystitis, painful bladder syndrome, prostatitis, urethritis, post-surgical pain, and kidney stones.

Exemplary active agents for treating or ameliorating one or more symptoms associated with inflammation of the urinary tract, the bladder, and/or the kidney(s) include, but are not limited to, lidocaine, amitriptyline, cimetidine, hydroxyzine, pentosan polysulfate, triamcinolone, cyclosporine A, glycosaminoglycans (e.g., chondroitin sulfate), sulfoxide, pentosan polysulfate sodium (PPS), dimethyl sulfoxide (DMSO), oxybutynin, mitomycin C, heparin, aurothiomalate, aurothioglucose, aurothiopropanol sulfonate, flavoxate, and ketorolac, and a combination thereof. Optionally, the anti-inflammatory active agents may be used in combination with a drug for pain, such as cannabinoids, CBD, a nonsteroidal anti-inflammatory drug (NSAID), diclofenac, ibuprofen and analogs thereof.

Optionally, the patient needs desquamative treatment. An exemplary active agent for treating or ameliorating one or more symptoms needing desaquamation is salicylic acid.

The amount of the one or more active agents required will vary from subject to subject according to their need. Typically, the amount of the active agents is effective to treat or ameliorate at least one of the one or more symptoms associated with inflammation of the urinary tract, the bladder, and/or the kidney(s).

b. Overactive Bladder and Bladder Incontinence

Optionally, the patient being treated may have one or more symptoms associated with overactive bladder, neurogenic bladder, bladder incontinence, and motility.

Exemplary active gents for treating or ameliorating one or more symptoms associated with overactive bladder, neurogenic bladder, bladder incontinence, and motility include, but are not limited to, antimuscarinic compounds, antispasmodic agents anticholinergic agents, botulinum toxin, onabotulinumtoxin A, beta-2 agonists, alpha adrenergics, anticonvulsants, norepinephrine uptake inhibitors, serotonin uptake inhibitors, calcium channel blockers, potassium channel openers, phosphodiesterase type 5 inhibitor (PDE5 inhibitors), muscle relaxants, apomorphine, darifenacin, tolterodine, oxybutynin, propiverine, trospium, solifenacin, mirabegron, cannabinoids, fesoterodine and analogs thereof, and a combination thereof. Any of the above described active agents can be used in combination with an anesthetic agent.

The amount of the one or more active agents required will vary from subject to subject according to their need. Typically, the amount of the active agents is effective to treat or ameliorate at least one of the one or more symptoms associated with overactive bladder, bladder incontinence, and motility. For example, the amount of the active agents is effective to reduce episodes of urge incontinence.

c. Bladder and Kidney Cancer

Optionally, the patient being treated may have bladder and/or kidney cancer, such as urothelial carcinoma, squamous cell carcinoma, noninvasive papillary carcinoma, adenocarcinoma, and squamous cell carcinoma.

Exemplary active agents for treating or ameliorating one or more symptoms associated with bladder and/or kidney cancer include, but are not limited to, antiproliferative agents, cytotoxic agents, chemotherapeutic agents, immunomodulatory, monoclonal antibody, a TNF inhibitor, an anti-leukin, kinase inhibitor, desquamative agents or a combination thereof, apaziquone, atezolizumab, avelumab, bavencio, cisplatin, doxorubicin, durvalumab, epirubicin, 5-FU (5-fluorouracil), gemcitabine, imfinzi, keytruda, methotrexate, mitomycin C, nivolumab, opdivo, pembrolizumab, pirarubicin, paclitaxel, tecentriq, thiotepa, valrubicin, valstar, valrubicin and analogs thereof, and a combination thereof.

The amount of the one or more active agents required will vary from subject to subject according to their need. Typically, the amount of the active agents is effective to kill or decrease the level of cancer cells associated with the bladder and/or kidney cancer.

d. Bladder and/or Kidney Pain

Optionally, the patient being treated may have bladder or kidney pain, neurogenic bladder, or interstitial cystitis.

Exemplary active agents for pain, bladder pain syndrome, or interstitial cystitis include, but are not limited to, anesthetic agents, analgesic agents, anti-inflamatory agents and combinations thereof, aminoamides, lidocaine base or lidocaine salt, procaine, articaine, benzocaine, bupivacaine, tramadol or tramadol salt, dibucaine, lontocaine, mepivacaine, prilocaine, ropivacaine, tanezumab, gabapentin, chloroprocaine, cocaine, cocaine analogues, proparacaine, tetracaine, cannabinoids, CBD, tetrahydrocannabinol (THC) and their combinations, NSAIDs, paracetamol, diclofenac, ibuprofen, naproxen, piroxicam, acetaminophen, flufenisal, indoprofen, indomethacin and analogs thereof.

Exemplary opioid agonists include, but are not limited to, benzylmorphine, buprenorphine, butorphanol, desomorphine, dextromoramide, dezocine, diampromide, diamorphone, dihydrocodeine, dihydromorphine, ethylmorphine, fentanyl, heroin, hydrocodone, hydromorphone, hydroxypethidine, isomethadone, methadone, morphine, myrophine, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol HCL, pharmaceutically acceptable salts thereof, and a combination thereof.

For kidney stones, the above described drug(s) may be selected to treat pain and/or to promote dissolution of renal stones.

The amount of the one or more active agents required will vary from subject to subject according to their need. Typically, the amount of the active agents is effective to reduce the level of or prevent bladder and/or kidney pain.

e. Urinary Tract, Bladder or Kidney Infection

Optionally, the patient being treated may have one or more symptoms associated with urinary tract, bladder, and/or kidney infection, such as urinary tract infections and bladder or kidney viral infections.

Exemplary active agents for treating or ameliorating one or more symptoms associated with bladder or urinary tract infections include, but are not limited to, antibiotics, amoxicillin, ceftriaxone, cephalexin, ciprofloxacin, fosfomycin, levofloxacin, minocycline, nitrofurantoin, trimethoprim and analogs thereof, sulfamethoxazole and analogs thereof.

Exemplary active agents for treating or ameliorating one or more symptoms associated with urinary tract, bladder, or kidney viral infection (e.g., adenoviral infection, BK virus, and Cytomegalovirus), include, but are not limited to, acyclovir, acidofovir, amantadine, rimantadine, morpholino oligos, double-stranded RNA activated caspase oligomerizer, and a combination thereof.

The amount of the one or more antibiotic and/or antimicrobial agents required will vary from subject to subject according to their need. Typically, the amount of the active agents is effective to treat or ameliorate at least one of the one or more symptoms associated with bladder and/or kidney infection.

f. Fibroids

Optionally, the patient being treated may have one or more symptoms associated with fibroids of the bladder.

Exemplary active agents for treating or ameliorating one or more symptoms associated with bladder fibroids include, but are not limited to, pentoxphylline, antiTNF, antiTGF agents, GnRH analogues, exogenous progestins, antiprogestins, selective estrogen receptor modulators, danazol, and NSAIDs, and a combination thereof.

The amount of the one or more active agents required will vary from subject to subject according to their need. Typically, the amount of the active agents is effective to treat or ameliorate at least one of the one or more symptoms associated with bladder fibroids.

g. Additional Pathologies and Medical Conditions_{[TW5][DT6]}

Optionally, the patient being treated may have one or more symptoms associated with the pathology or a medical condition associated with urinary tract, such as pelvic pain, bladder rupture, bladder tamponade, urinary retention, hematuria, hydronephrosis in newborns, vesicoureteral reflux (VUR), prolapsed bladder or cystocele, bladder stones, a dynamic ureteral segments, ureteral stricture, or erectile dysfunction.

Active agents for treating or ameliorating one or more symptoms associated with the above described pathologies and medical conditions are known. The amount of the one or more active agents required will vary from subject to subject according to their need. Typically, the amount of the active agents is effective to treat or ameliorate at least one of the one or more symptoms associated with one or more of the above described pathologies and medical conditions.

The disclosed formulations and methods can be further understood through the following numbered paragraphs.

1. A liquid formulation for instillation into the urinary tract, bladder, or kidney(s) comprising:
an alcohol, optionally more than one alcohol, optionally wherein the alcohol is a short-chain alcohol;
one or more polymers; and
one or more active agents,

wherein when in contact with urine, the one or more polymers precipitate from the formulation and entrap the one or more active agents, forming a mass in the urinary tract, bladder, or kidney(s), and

wherein the mass releases the one or more agents over an extended period of time.

2. The formulation of paragraph 1, wherein the one or more polymers are soluble in the one or more alcohols and practically insoluble in water or an aqueous solution at a pH≤6.
3. A liquid formulation for instillation into the urinary tract, bladder, or kidney(s) comprising:
an alcohol, optionally more than one alcohol, optionally wherein the alcohol is a short-chain alcohol;
one or more polymers; and
one or more active agents,

wherein the one or more polymers are soluble in the alcohol and practically insoluble in water or an aqueous solution at a pH≤6.

4. The formulation of any one of paragraphs 1-3, wherein the alcohol is a short-chain alcohol, preferably a C₂-C₄ alcohol, such as ethanol or propylene glycol.
5. The formulation of any one of paragraphs 1-4, wherein the formulation further comprises water from 0% to 20% (w/w) of the formulation.
6. The formulation of any one of paragraphs 1-5, wherein the one or more polymers comprise ethylcellulose or an acrylate polymer, or a combination thereof.
7. The formulation of any one of paragraphs 1-6, wherein the one or more active agents comprise a therapeutic agent, a prophylactic agent, or a diagnostic agent, or a combination thereof.
8. The formulation of any one of paragraphs 1-7, wherein the one or more polymers comprise ethylcellulose and wherein the ethylcellulose has an ethoxyl content from 40 wt % to 49 wt %, from 45 wt % to 49 wt %, or from 46 wt % to 48 wt %.
9. The formulation of any one of paragraphs 1-8, wherein the one or more polymers comprise an acrylate polymer, and wherein the acrylate polymer is poly(methacrylate), poly(ethylacrylate), a copolymer thereof, or polycarbophil.
10. The formulation of any one of paragraphs 1-9, wherein the one or more polymers comprise an acrylate polymer, and wherein the acrylate polymer is poly(methyl methacrylate), poly(ethyl methacrylate), poly(N,N-dimethylaminoethyl methacrylate), poly(butyl methacrylate), or poly(ethylacrylate), a copolymer thereof, or polycarbophil.
11. The formulation of any one of paragraphs 1-10, further comprising one or more additives.
12. The formulation of paragraph 11, wherein the one or more additives comprise molecules, oligomers, and/or polymers that are water- and alcohol-dispersible.
13. The formulation of paragraph 11 or paragraph 12, wherein the one or more additives comprise one or more pharmaceutically acceptable excipients selected from the group consisting of plasticizers, viscosity modifiers, surfactants, a pH buffering agent, permeation enhancers, diluents, lubricants, preservatives, antioxidants, binders, disintegrators, and stabilizers, or a combination thereof.
14. The formulation of paragraph 13, wherein the surfactant has a hydrophile-lipophile balance (HLB) from 7 to 10, or from 1.5 to 6, or higher than 10.
15. The formulation of paragraph 13 or paragraph 14, wherein the one or more additives comprise one or more surfactants selected from the group consisting of sorbitan esters, polysorbates, lecithin, and phospholipids, or a combination thereof.
16. The formulation of paragraph 15, wherein the phospholipids are selected from the group consisting of saturated phospholipids, soy phospholipid, egg phospholipid, and phosphatidylcholine, or a combination thereof.
17. The formulation of any one of paragraphs 11-16, wherein the one or more additives comprise one or more water- and alcohol-dispersible polymers selected from the group consisting of hydroxypropylcellulose, polyacrylic acid polymer, polyvinyl acetate phthalate, and polyvinylpyrrolidone, or a combination thereof.
18. The formulation of any one of paragraphs 1-17, wherein the total concentration of the one or more active agents is in a range from 0.005 wt % to 20 wt %, from 0.005 wt % to 15 wt %, from 0.005% to 10% w/w, from 0.005% to 5% w/w, from 0.005% to 1% w/w, from 0.005% to 0.5% w/w, from 0.005% to 0.1% w/w, from 0.005% to 0.05% w/w, from 0.01% to 20% w/w, from 0.01% to 15% w/w, from 0.01% to 10% w/w, from 0.01% to 5% w/w, from 0.01% to 1% w/w, from 0.05% to 20% w/w, from 0.05% to 15% w/w, from 0.05% to 10% w/w, from 0.05% to 5% w/w, from 0.05% to 1% w/w, from 0.1% to 20% w/w, from 0.1% to 15% w/w, from 0.1 wt % to 10 wt %, or from 0.1 to 5 wt % of the formulation.
19. The formulation of any one of paragraphs 1-18, wherein the total concentration of the one or more polymers is in a range from 1 wt % to 20 wt %, from 2 wt % to 20 wt %, from 1 wt % to 10 wt %, from 2 wt % to 8 wt %, or from 2 wt % to 7 wt % of the formulation.
20. The formulation of any one of paragraphs 1-19, wherein the total concentration of the alcohol is from 10% to 90% w/w, from 12% to 50% w/w, from 20% to 80% w/w, from 30% to 75% w/w, from 40% to 90% w/w, from 50% to 95% w/w, or from 60% to 98% w/w of the formulation.
21. The formulation of any one of paragraphs 1-20, wherein the alcohol comprises ethanol and wherein the ethanol is present in an amount up to 49% w/w, up to 35% w/w, from 12% to 35% w/w, from 12% to 90% w/w, from 20% to 80% w/w, from 30% to 75% w/w, from 40% to 90% w/w, from 50% to 95% w/w, or from 60% to 98% w/w of the formulation.
22. The formulation of paragraph 21, wherein the ethanol is ethanol absolute or ethanol having a purity from 80% to 99% v/v.
23. The formulation of any one of paragraphs 1-22, wherein the alcohol comprise a glycol and wherein the glycol is present in an amount up to 96% w/w, from 12% to 96% w/w, from 12% to 45% w/w, from 20% to 60% w/w, or from 1% to 55% w/w of the formulation.
24. The formulation of paragraph 23, wherein the glycol is propylene glycol.
25. The formulation of any one of paragraphs 11-24, wherein the total concentration of the one or more additives is in a range from 0.1 wt % to 20 wt %, from, from 0.1 wt % to 15 wt %, from 1 wt % to 12 wt %, from 1 wt % to 10 wt %, from 1 wt % and 15 wt %, from 2 wt % to 20 wt %, from 2 wt % to 15 wt %, from 2 wt % to 10 wt %, from 3 wt % to 20 wt %, from 3 wt % to 15 wt %, or from 3 wt % to 10 wt % of the formulation.
26. The formulation of any one of paragraphs 13-25, wherein the total concentration of the one or more surfactants is in a range up to 20% w/w, up to 15% w/w, up to 8% w/w, from 0.1% to 10% w/w, or from 2% to 5% w/w of the formulation.
27. The formulation of any one of paragraphs 12-26, wherein the total concentration of the one or more water- and alcohol-dispersible polymers is in a range up to 20% w/w, 15% w/w, up to 8% w/w, from 0.1% to 10% w/w, from 2% to 4% w/w, from 1% to 5% w/w, or from 2% to 6% w/w of the formulation.
28. A pre-filled syringe or container comprising the formulation of any one of paragraphs 1-27.
29. A method for treating or ameliorating one or more symptoms associated with a disorder or disease in or affecting a patient's urinary tract, bladder, and/or kidney(s) comprising:

(i) inserting a catheter, a cystoscope, or an ureteroscope into the urinary tract, bladder, and/or kidney(s) of the patient, and

(ii) instilling the formulation of any one of paragraphs 1 to 27 into the urinary tract, bladder, and/or kidney(s) through the catheter, the cystoscope, or the ureteroscope,

wherein following step (ii), the formulation forms a mass in the patient's urine and the one or more active agents are entrapped in the mass.

30. A method for providing prolonged release of an active agent into a patient's urinary tract, bladder, and/or kidney(s) comprising:

(i) inserting a catheter, a cystoscope, or an ureteroscope into the urinary tract, bladder, and/or kidney(s) of the patient, and

(ii) instilling the formulation of any one of paragraphs 1 to 27 into the urinary tract, bladder, and/or kidney(s) through the catheter, the cystoscope, or the ureteroscope,

wherein following step (ii), the formulation forms a mass in the patient's urine and the one or more active agents are entrapped in the mass.

31. The method of paragraph 29 or paragraph 30, wherein the mass has a largest dimension from 0.5 cm to 20 cm, from 8 mm to 12 cm, or from 2 cm to 7 cm.
32. The method of any one of paragraphs 29-31, wherein the formulation has an instillation volume from 0.5 mL to 120 mL, from 4 mL to 100 mL, from 2 mL to 60 mL, from 5 mL to 40 mL, from 1 mL to 30 mL, from 1 mL to 10 mL, or from 1 mL to 5 mL.
33. The method of any one of paragraphs 29-32, further comprising repeating step (ii).
34. The method of paragraph 33, further comprising repeating step (ii) every hour, every 2 hours, every 5 hours, every 8 hours, every day, every 2 days, every 3 days, every 5 days, every 7 days, every 10 days, every 30 days.
35. The method of paragraph 33 or paragraph 34, wherein step (ii) is repeated with the same formulation or a different formulation.
36. The method of any one of paragraphs 29-35, wherein following step (ii), the active agent is released into the urine for up to 1 hour, up to 2 hours, up to 5 hours, up to 8 hours, up to 24 hours, up to 48 hours, up to 3 days, up to 4 days, up to 5 days, up to 6 days, up to 7 days, up to 15 days, up to 30 days, or more than 30 days.
37. The method of any one of paragraphs 29-36, wherein following step (ii), the formulation forms the mass in the patient's urine upon contact with the urine.
38. The method of any one of paragraphs 29-37, wherein the mass floats on the surface of the urine or is immersed in the urine.
39. The method of any one of paragraphs 29-38, further comprising

step (iii) adjusting the release rate of the one or more active agents from the mass prior to step (i), subsequent to step (i) and prior to step (ii), and/or subsequent to step (ii).

40. The method of paragraph 39, wherein step (iii) comprises

instilling a basic solution into the urinary tract, bladder, and/or kidney(s), administering bicarbonate medications, or consuming food associated with raising the urine pH, or a combination thereof.

41. The method of any one of paragraphs 29-40, wherein the patient has one or more symptoms associated with inflammation of the urinary tract, bladder, and/or kidney(s), and wherein the active agent is in an effective amount to treat or ameliorate at least one of the one or more symptoms associated with inflammation of the urinary tract, bladder, and/or kidney(s).
42. The method of any one of paragraphs 29-40, wherein the patient has one or more symptoms associated with overactive bladder and/or neurogenic bladder, and wherein the active agent is in an effective amount to treat or ameliorate at least one of the one or more symptoms associated with overactive bladder and/or neurogenic bladder.
43. The method of any one of paragraphs 29-40, wherein the patient has bladder and/or kidney cancer, and wherein the active agent is in an effective amount to kill or decrease the level of cancer cells associated with the bladder and/or kidney cancer.
44. The method of any one of paragraphs 29-40, wherein the patient has bladder and/or kidney pain, and wherein the active agent is in an effective amount to reduce the level of or prevent bladder and/or kidney pain.
45. The method of any one of paragraphs 29-40, wherein the patient has one or more symptoms associated with infection of the urinary tract, bladder, and/or kidney(s), and wherein the active agent is in an effective amount to treat or ameliorate at least one of the one or more symptoms associated with the infection.
46. The method of any one of paragraphs 29-40, wherein the patient has one or more symptoms associated with interstitial cystitis of the bladder, and wherein the active agent is in an effective amount to treat or ameliorate at least one of the one or more symptoms associated with interstitial cystitis of the bladder.
47. The method of any one of paragraphs 29-46, wherein the formulation provides prolonged release of the one or more active agents compared to a control, wherein the control comprises the one or more active agents at the same concentration (w/w) in the same alcohol as the formulation.
48. The method of paragraph 47, wherein the control does not contain the one or more one or more polymers.
49. The method of any one of paragraphs 47-48, wherein the formulation further comprises one or more additives and wherein the control does not contain the one or more additives.

The present invention will be further understood by reference to the following non-limiting examples.

### EXAMPLES

### Example 1

| | % w/w |
|---|---|
| Lidocaine | 1 |
| Eudragit ® S100 | 7 |
| Ethanol abs. | 40 |
| Propylene Glycol | 47 |
| Span ® 20 | 5 |

Preparation

In a well-covered vessel, Eudragit® was added to ethanol and mixed. Lidocaine was then added to the polymer solution with mixing. Span® 20 was further added and mixed. Finally, propylene glycol was added and mixed. The final formulation was mixed.

Results

As shown in FIG. 2B, a mass 10″ was formed in urine upon contact of the formulation with urine. The formed mass 10″ was immersed in the bulk of the urine.

### Example 2

| | % w/w |
|---|---|
| Lidocaine | 1 |
| Eudragit ® S100 | 5 |
| Ethylcellulose | 5 |
| Ethanol abs. | 40 |
| Propylene Glycol | 49 |

Preparation

In a well-covered vessel, Eudragit® was added to ethanol with continuing mixing until a clear solution was obtained. Ethylcellulose was then added slowly and mixed until a clear solution was obtained. Lidocaine was then added to the polymer solution with mixing. Finally, propylene glycol was added. The final formulation was mixed.

### Example 3

| | % w/w |
|---|---|
| Lidocaine base | 4 |
| Ethylcellulose 100 | 6 |
| Ethanol abs. | 90 |

Preparation

In a well-covered vessel, Ethylcellulose was added to ethanol and mixed. Lidocaine was then added to the polymer solution with mixing. The final formulation was mixed. 5 mL of the solution where transferred into a syringe and injected into a glass vial containing 50 mL human urine, forming instantly in situ “Capture” mass body.

### Example 4

| | % w/w |
|---|---|
| Lidocaine base | 3 |
| Lidocaine HCL | 2 |
| Ethylcellulose 100 | 5 |
| Ethanol abs. | 85 |
| Span ® 20 | 5 |

Preparation

In a well-covered vessel, Ethylcellulose was added to ethanol and mixed until a clear solution was obtained. Span®20 was then added with mixing. Lidocaine base was added to the above solution with mixing. Then Lidocaine HCl was added and mixed. The final formulation was mixed forming a solution.

2 mL of the formulation where transferred into a syringe and injected into a glass vial containing 20 mL human urine, forming instantly in situ a mass body.

### Example 5

| | % w/w |
|---|---|
| Hydrocortisone | 0.5 |
| Lidocaine base | 3 |
| Lidocaine HCL | 3 |
| Ethylcellulose 100 | 7.5 |
| Span ® 20 | 5 |
| Ethanol 96 | 86 |

Preparation

In a well-covered vessel, Ethylcellulose was added to ethanol with continuous mixture until a clear solution was obtained. Span® 20 was then added to the above solution with mixing. Hydrocortisone and Lidocaine were added and mixed. Lidocaine HCl was added and mixed in the above formulation. The final formulation was mixed.

### Example 6

| | % w/w |
|---|---|
| Hydrocortisone | 0.5 |
| Ethylcellulose 100 | 5.5 |
| Sorbitan ester (Span ® 20) | 5 |
| Ethanol abs. | 89 |

Preparation

In a well-covered vessel, Ethylcellulose was added to ethanol and mixed. Hydrocortisone was then added to the polymer solution with mixing. Span® 20 was then added and mixed. The final formulation was mixed.

### Example 7

| | % w/w |
|---|---|
| Lidocaine | 1 |
| Eudragit ® S100 | 5 |
| Ethanol abs. | 50 |
| Propylene Glycol | 39 |
| Span ® 20 | 5 |

Preparation

In a well-covered vessel, Eudragit® was added to ethanol and mixed. Lidocaine was then added to the polymer solution with mixing. Span® 20 was then added and mixed. Finally, propylene glycol was added. The final formulation was mixed.

### Example 8

| | % w/w |
|---|---|
| Lidocaine | 5 |
| Ethylcellulose | 4.2 |
| Eudragit ® S100 | 4.2 |
| Span ® 20 | 12.4 |
| Ethanol abs | 74.2 |

Preparation

In a well-covered vessel, Ethylcellulose was added to ethanol and mixed until dissolution. Then Eudragit® was added with continuous mixing. Lidocaine was then added to the polymer solution with mixing. Span® 20 was then added and mixed. The final formulation was mixed.

### Example 9

| | % w/w |
|---|---|
| Bupivacaine | 1 |
| Eudragit ® S100 | 7 |
| Sorbitan ester HLB 7-8.5 | 12.8 |
| Carbopol ® 974P | 0.2 |
| Propylene glycol | 29 |
| Ethanol abs | 50 |

In a well-covered vessel, Eudragit® was added to ethanol and mixed.

Bupivacaine was then added to the polymer solution with mixing. The amphiphile, Sorbitan ester HLB 7-8.5, was added and mixed. Propylene glycol was added and Carbopol® was slowly dispersed with mixing on the surface of the liquid. The final formulation was mixed from time to time while left covered until the dissolution of the Carbopol®. The final formulation was mixed.

### Example 10

| | % w/w |
|---|---|
| Ibuprofen | 5 |
| Ethylcellulose | 5 |
| Eudragit ® S100 | 4 |
| Span ® 20 | 12 |
| Propylene glycol | 15 |
| Ethanol | 59 |

Preparation

In a well-covered vessel, Ethylcellulose was added to ethanol and mixed, then Eudragit® was added with continuous mixing to form a polymer solution. Ibuprofen was then added to the polymer solution with mixing. Span® 20 was added and mixed. Propylene glycol was added with mixing. The final formulation was mixed.

### Example 11

| | % w/w |
|---|---|
| Ibuprofen | 7 |
| Ethylcellulose | 4 |
| Eudragit ® S100 | 7 |
| Span ® 20 | 12 |
| Propylene Glycol | 20 |
| Ethanol Abs. | 50 |

Preparation

In a well-covered vessel, Ethylcellulose was added to ethanol and mixed, then Eudragit® was added with continuous mixing. Ibuprofen was then added to the polymers solution with mixing. Span® 20 was added and mixed. Propylene glycol was added with mixing. The final formulation was mixed.

### Example 12

| | % w/w |
|---|---|
| Cannabidiol | 0.5 |
| Oxybutynin | 0.5 |
| Ethylcellulose 100 | 7 |
| Span ® 20 | 11 |
| Ethanol abs. | 81 |

Preparation

In a well-covered vessel, Ethylcellulose was added to ethanol and mixed. Oxybutinin and Cannabidiol were then added to the polymer solution with mixing. Span® 20 was added and mixed. The final formulation was mixed.

### Example 13

| | % w/w |
|---|---|
| Naproxen | 5 |
| Ethylcellulose 100 | 3 |
| Eudragit ® S100 | 8 |
| Span ® 20 | 15 |
| Klucel ™ HPC | 5 |
| Propylene glycol | 14 |
| Ethanol abs. | 50 |

Preparation

In a well-covered vessel, Eudragit® was added to ethanol and mixed, then Ethylcellulose was added with continuous mixing. Naproxen was added to the polymers solution with mixing. Propylene glycol was added with mixing. Span® 20 was added and mixed. Klucel™ was added with mixing and the formulation was left until Klucel™ dissolved. The final formulation was mixed.

### Example 14

| | % w/w |
|---|---|
| Triamcinolone | 0.1 |
| Ethylcellulose 100 | 7 |
| Span ® 20 | 15 |
| Tween ® 80 | 7 |
| Ethanol abs. | 70.9 |

Preparation

In a well-covered vessel, Ethylcellulose was added to ethanol and mixed until dissolved. Tween 80® was then added with mixing until dissolved. Triamcinolone was then added and mixed to dissolve. Span® 20 was then added and mixed. The final formulation was mixed.

### Example 15

| | % w/w |
|---|---|
| Triamcinolone | 0.2 |
| Eudragit ® S100 | 7.8 |
| Lecithin | 7 |
| Propylene glycol | 40 |
| Ethanol abs. | 45 |

Preparation

In a well-covered vessel, Lecithin was added to ethanol and mixed until dissolved. Eudragit® was then added with mixing until dissolved. Triamcinolone was then added and mixed to dissolve. Propylene glycol are added and mixed. The final formulation was mixed.

### Example 16

| | % w/w |
|---|---|
| Triamcinolone | 1 |
| Eudragit ® S100 | 10 |
| Span ® 20 | 14 |
| Lecithin | 2 |
| Propylene glycol | 33 |
| Ethanol abs. | 40 |

Preparation

In a well-covered vessel, Lecithin was added to ethanol and mixed until dissolved. Eudragit® was then added with mixing until dissolved. Triamcinolone was then added and mixed to dissolve. Span® 20 and propylene glycol are added and mixed. The final formulation was mixed.

### Example 17

| | % w/w |
|---|---|
| Gentamycin | 2.5 |
| Cannabidiol | 0.5 |
| Eudragit ® E100 | 10 |
| Hydroxypropylcellulose (HPC) | 3 |
| Propylene glycol | 35 |
| Surfactant HLB 8-9 | 10 |
| Ethanol 96 | 38 |

Preparation

In a well-covered vessel, Eudragit® was added to ethanol and mixed. Cannabidiol was added with mixing. Propylene glycol was then added to the polymer solution with mixing. The surfactant was then added and mixed. HPC was added with mixing and the formulation was left until HPC dissolved. The formulation was mixed. Gentamycin was added with mixing. The final formulation was mixed.

### Example 18

| | % w/w |
|---|---|
| Cannabidiol | 0.3 |
| Eudragit ® E100 | 7.7 |
| Span ® 20 | 10 |
| Propylene glycol | 32 |
| Ethanol abs. | 50 |

Preparation

In a well-covered vessel, Eudragit® was added to ethanol and mixed. Cannabidiol was then added to the polymer solution with mixing. Span® 20 was then added and mixed. Finally, propylene glycol was added. The final formulation was mixed.

### Example 19

| | % w/w |
|---|---|
| Cannabidiol | 0.3 |
| Lidocaine | 1 |
| Eudragit ® S100 | 7.7 |
| Span ® 20 | 10 |
| Propylene glycol | 31 |
| Ethanol abs. | 50 |

Preparation

In a well-covered vessel, Eudragit® was added to ethanol and mixed. Cannabidiol and Lidocaine were then added to the polymer solution with mixing. Span® 20 was added and mixed. Finally, propylene glycol was added. The final formulation was mixed.

### Example 20

| | % w/w |
|---|---|
| Cannabidiol | 0.5 |
| Tetrahydrocannabinol (THC) | 0.1 |
| Eudragit ® S100 | 5.4 |
| Span ® 20 | 10 |
| Propylene glycol | 34 |
| Ethanol abs. | 50 |

Preparation

In a well-covered vessel, Eudragit® was added to ethanol and mixed. Cannabidiol and THC were then added to the polymer solution with mixing. Propylene glycol was added and mixed. Finally, Span® 20 was added. The final formulation was mixed.

### Example 21

| | % w/w |
|---|---|
| Tetrahydrocannabinol (THC) | 0.2 |
| Eudragit ® S100 | 5.8 |
| Span ® 20 | 5 |
| Hydroxypropylcellulose | 3 |
| Propylene glycol | 26 |
| Ethanol abs. | 60 |

Preparation

In a well-covered vessel, Eudragit® was added to ethanol and mixed to dissolve. THC was then added to the polymer solution with mixing. Propylene glycol was added with mixing. Span® 20 was then added and mixed. Hydroxypropylcellulose was added with mixing and the formulation was left until it dissolved. The final formulation was mixed.

### Example 22 Formulations for Incorporation of Active Agent

| | % w/w |
|---|---|
| Eudragit ® S100 | 7 |
| Ethanol 96 | 50 |
| Span ® 20 | 15 |
| Hydroxypropylcellulose | 5 |
| Propylene Glycol | 23 |

The above formulation can be used to incorporate diagnostic probes, radioactive agents, fluorescent probes and other agents for diagnosis/action in the bladder.

The method for forming the above formulation included dissolving the polymers in ethanol using well-closed vessels and overhead mixers (Heldolph), addition of other ingredients (Span®, propylene glycol) with mixing. The hydroxypropylcellulose polymer was dispersed at the end and left to dissolve. The final formulation was then mixed.

### Example 23 Formulations for Incorporation of Active Agent

| | % w/w |
|---|---|
| Ethylcellulose 100 | 7 |
| Ethanol 96 | 73 |
| Span ® 20 | 15 |
| Hydroxypropylcellulose | 5 |

The above formulation can be used to incorporate diagnostic probes, radioactive agents, fluorescent probes and other agents for diagnosis/action in the bladder.

The preparation methods for the above formulation include the dissolution of polymers in ethanol using well closed vessels and overhead mixers (Heldolph), addition of other ingredients (Span®) with mixing. The hydroxypropylcellulose polymer was dispersed at the end and left to dissolve. The final formulation was then mixed.

### Example 24

| | % w/w |
|---|---|
| Mirabegron | 0.05 |
| Eudragit ® S100 | 8 |
| Span ® 20 | 9.85 |
| Propylene Glycol | 32 |
| Ethanol 95 | 50 |

Preparation

In a well-covered vessel, Mirabegron was added to ethanol and mixed. Eudragit® was then added to the drug solution with mixing. Propylene glycol was added. Finally, Span® 20 was added and mixed. The final formulation was mixed.

### Example 25

| | % w/w |
|---|---|
| Mirabegron | 0.2 |
| Eudragit ® S100 | 7.5 |
| Span ® 20 | 2.3 |
| Propylene Glycol | 45 |
| Ethanol 95 | 45 |

Preparation

In a well-covered vessel, Mirabegron was added to ethanol and mixed. Eudragit® was added with continues mixing until a clear solution was obtained. The final formulation was mixed.

### Example 26

| | % w/w |
|---|---|
| Oxybutynin | 0.1 |
| Eudragit ® S100 | 8 |
| Span ® 20 | 9.9 |
| Propylene Glycol | 15 |
| HPC | 2 |
| Ethanol abs. | 65 |

Preparation

In a well-covered vessel, Oxybutynin was added to ethanol and mixed. Eudragit® was then added with mixing. Propylene glycol was added with mixing. Span® 20 was added and mixed. Hydroxypropylcellulose was added with mixing and the formulation was left until it dissolved. The final formulation was mixed.

### Example 27

| | % w/w |
|---|---|
| Oxybutynin | 0.2 |
| Eudragit ® S100 | 8.8 |
| Span ® 20 | 9 |
| Propylene Glycol | 21 |
| HPC | 1 |
| Ethanol abs. | 60 |

Preparation

In a well-covered vessel, Oxybutynin was added to ethanol and mixed. Eudragit® was then added with mixing. Propylene glycol was added with mixing. Span® 20 was added and mixed. Hydroxypropylcellulose was added with mixing and the formulation was left until it dissolved. The final formulation was mixed.

### Example 28

| | % w/w |
|---|---|
| Oxybutynin | 0.5 |
| Eudragit ® S100 | 6.5 |
| Phospholipid | 6 |
| Propylene Glycol | 65 |
| HPC | 2 |
| Ethanol abs. | 20 |

Preparation

In a well-covered vessel, Phospholipid was added to ethanol and mixed to dissolve. Eudragit® was then added to ethanol and mixed until dissolved. Propylene glycol was added with mixing. Oxybutynin was then added with mixing. Hydroxypropylcellulose was added with mixing and the formulation was left until it dissolved. The final formulation was mixed.

### Example 29

| | % w/w |
|---|---|
| Doxorubicin | 0.1 |
| Eudragit ® RS100 | 7 |
| Span ® 20 | 5.9 |
| Propylene Glycol | 37 |
| Ethanol 96 | 50 |

Preparation

In a well-covered vessel, Eudragit® was added to ethanol and mixed. Propylene glycol and Span® are added. Finally, Doxorubicin was added with mixing. The final formulation was mixed.

### Example 30

| | % w/w |
|---|---|
| Doxorubicin | 0.2 |
| Eudragit ® RS100 | 8 |
| Span ® 20 | 6.8 |
| Propylene Glycol | 35 |
| Ethanol 96 | 50 |

Preparation

In a well-covered vessel, Eudragit® was added to ethanol and mixed. Propylene glycol and Span® are added. Finally, Doxorubicin was added with mixing. The final formulation was mixed.

### Example 31

| | % w/w |
|---|---|
| Doxorubicin | 0.1 |
| Eudragit ® L100 | 7 |
| Sorbitan ester HLB 8.6 | 5.9 |
| Hydroxypropylcellulose | 2 |
| Propylene Glycol | 35 |
| Ethanol 96 | 50 |

Preparation

In a well-covered vessel, Eudragit® was added to ethanol and mixed. Propylene glycol was added with mixing. Sorbitan ester was added and mixed. Hydroxypropylcellulose was added with mixing and the formulation was left until it dissolved. To the final formulation, Doxorubicin was added with mixing.

### Example 32

| | % w/w |
|---|---|
| Doxorubicin | 0.3 |
| Eudragit ® L100 | 5.2 |
| Sorbitan ester HLB 8.6 | 6.5 |
| Hydroxypropylcellulose | 2 |
| Propylene Glycol | 36 |
| Ethanol 95 | 50 |

Preparation

In a well-covered vessel, Eudragit® was added to ethanol and mixed. Propylene glycol was added with mixing. Sorbitan ester was added and mixed. Hydroxypropylcellulose was added with mixing and the formulation was left until it dissolved. To the final formulation, Doxorubicin was added with mixing.

### Example 33

| | % w/w |
|---|---|
| Thiotepa | 0.1 |
| Eudragit ® RS100 | 5.9 |
| Span ® 20 | 6 |
| Propylene Glycol | 38 |
| Ethanol | 50 |

Preparation

In a well-covered vessel, Thiotepa was added to ethanol and mixed. Eudragit® was then added to the drug solution with mixing. Propylene glycol was added. Finally, Span® 20 was added and mixed. The final formulation was mixed.

### Example 34

| | % w/w |
|---|---|
| Thiotepa | 0.5 |
| Eudragit ® RS100 | 6.5 |
| Span ® 20 | 6 |
| Propylene Glycol | 37 |
| Ethanol | 50 |

Preparation

In a well-covered vessel, Thiotepa was added to ethanol and mixed. Eudragit® was then added to the drug solution with mixing. Propylene glycol was added. Finally, Span® 20 was added and mixed. The final formulation was mixed.

### Example 35

| | % w/w |
|---|---|
| Tramadol HCl | 1 |
| Eudragit ® E100 | 7 |
| Propylene Glycol | 42 |
| Ethanol | 50 |

Preparation

In a well-covered vessel, Eudragit® was added to ethanol and mixed. Tramadol was then dissolved in the polymer solution with mixing. Propylene glycol was added and mixed. The final formulation was mixed.

Results

As shown in FIG. 2A, a mass 10′ was formed in urine upon contact of the formulation with urine. The formed mass 10′ was floating on the surface of the urine.

### Example 36

| | % w/w |
|---|---|
| Mirabegron | 0.1 |
| Eudragit ® S100 | 5 |
| Span ® 20 | 5 |
| Propylene Glycol | 45 |
| Ethanol | 50 |

Preparation

In a well-covered vessel, Mirabegron was added to ethanol and mixed. Eudragit® was then added to the drug solution with mixing. Propylene glycol and Span® 20 are added with mixing. The final formulation was mixed.

### Example 37

| | % w/w |
|---|---|
| Gemcitabine | 5 |
| Ethylcellulose 100 | 7 |
| Span ® 20 | 10 |
| Ethanol | 78 |

Preparation

In a well-covered vessel, Span® 20 was added to ethanol with mixing until dissolved. Then Ethylcellulose was added with continuous mixing. Gemcitabine was then added to the final formulation with mixing. The final formulation was mixed.

### Example 38

| | % w/w |
|---|---|
| Lidocaine | 5 |
| Ethylcellulose 100 | 5 |
| Eudragit ® S100 | 3 |
| Span ® 20 | 5 |
| Ethanol Abs | 82 |

Preparation

In a well-covered vessel, Ethylcellulose was added to ethanol and mixed until dissolved; then Eudragit® was added slowly with continuous mixing. Lidocaine then was added to the polymers solution with mixing. Span®20 was added and mixed. The final formulation was mixed.

### Example 39

| | % w/w |
|---|---|
| Tramadol HCl | 10 |
| Eudragit ® S100 | 8 |
| Hydroxypropylcellulose | 4 |
| Span ® 20 | 12 |
| Propylene glycol | 26 |
| Ethanol | 40 |

Preparation

In a well-covered vessel, Eudragit® was added to ethanol and mixed to dissolution. Tramadol was then added to the polymer solution with mixing. Propylene glycol was added with mixing. Span® 20 was then added and mixed. Hydroxypropylcellulose was added with mixing and the formulation was left until HPC dissolved. The final formulation was mixed.

### Example 40

| | % w/w |
|---|---|
| Tramadol HCl | 10 |
| Eudragit ® S100 | 8 |
| Hydroxypropylcellulose | 2 |
| Span ® 20 | 8 |
| Benzyl alcohol | 1 |
| Propylene glycol | 21 |
| Ethanol | 50 |

Preparation

In a well-covered vessel, Eudragit® was added to ethanol and mixed until dissolved. Tramadol was then added to the polymer solution with mixing. Propylene glycol was added with mixing. Span® 20 was added and mixed. Benzyl alcohol was added with mixing. Hydroxypropylcellulose was then added with mixing and the formulation was left until HPC dissolved. The final formulation was mixed.

### Example 41

| | % w/w |
|---|---|
| Ciprofloxacin | 5 |
| Eudragit ® S100 | 8 |
| Hydroxypropylcellulose | 2 |
| Span ® 20 | 15 |
| Propylene glycol | 25 |
| Ethanol | 45 |

Preparation

In a well-covered vessel, Eudragit® was added to ethanol and mixed until dissolved. Propylene glycol was added with mixing. Span® 20 was added and mixed. Hydroxypropylcellulose was added with mixing and the formulation was left until it dissolved. The formulation was mixed. Ciprofloxacin was added to the final formulation with mixing.

### Example 42

| | % w/w |
|---|---|
| Diclofenac sodium | 5 |
| Eudragit ® S100 | 6 |
| Hydroxypropylcellulose | 0.5 |
| Span ® 20 | 11.5 |
| Propylene glycol | 22 |
| Ethanol | 65 |

Preparation

In a well-covered vessel, Eudragit® was added to ethanol and mixed to dissolution. Diclofenac sodium was then added to the polymer solution with mixing. Propylene glycol was added with mixing. Span® 20 was added and mixed. Hydroxypropylcellulose was added with mixing and the formulation was left until HPC dissolved. The final formulation was mixed.

### Example 43

| | % w/w |
|---|---|
| Oxybutynin | 0.5 |
| Eudragit ® S100 | 6 |
| Phospholipid | 6.5 |
| Propylene Glycol | 55 |
| HPC | 2 |
| Ethanol abs. | 30 |

Preparation

In a well-covered vessel, Phospholipid was added to ethanol and mixed to dissolve. Eudragit® was then added to ethanol and mixed until dissolved. Propylene glycol was added with mixing. Oxybutynin was then added with mixing. Hydroxypropylcellulose was added with mixing and the formulation was left until it dissolved. The final formulation was mixed.

### Example 44

| | % w/w |
|---|---|
| Oxybutynin | 0.6 |
| Eudragit ® S100 | 6 |
| Phospholipid | 6 |
| Propylene Glycol | 44 |
| HPC | 2 |
| Vitamin E | 0.4 |
| Benzyl alcohol | 1 |
| Ethanol abs. | 40 |

Preparation

In a well-covered vessel, phospholipid was added to ethanol and mixed to dissolve. Eudragit® was then added to ethanol and mixed until dissolved. Vitamin E was added and mixed. Propylene glycol was added with mixing. Oxybutynin was then added with mixing. Benzyl alcohol was added with mixing. Hydroxypropylcellulose was added with mixing and the formulation was left until it dissolved. The final formulation was mixed.

### Example 45

| | % w/w |
|---|---|
| Mitomycin C | 0.4 |
| Eudragit ® S100 | 7.6 |
| Span ® 20 | 2 |
| Propylene Glycol | 54 |
| HPC | 2 |
| Ethanol abs. | 32 |

Preparation

In a well-covered vessel, phospholipid was added to ethanol and mixed to dissolve. Eudragit® was then added to ethanol and mixed until dissolved. Propylene glycol was added with mixing. Mitomycin was then added with mixing. Hydroxypropylcellulose was added with mixing and the formulation was left until it dissolved. The final formulation was mixed.

### Example 46

| | % w/w |
|---|---|
| Mitomycin C | 0.5 |
| Eudragit ® S100 | 6.5 |
| Phospholipid | 8 |
| Propylene Glycol | 37 |
| Ethanol abs. | 48 |

Preparation

In a well-covered vessel, phospholipid was added to ethanol and mixed to dissolve. Eudragit® was then added to ethanol and mixed until dissolved. Propylene glycol was added with mixing. Mitomycin C was then added with mixing. The final formulation was mixed.

### Example 47

| | % w/w |
|---|---|
| Mitomycin C | 0.2 |
| Eudragit ® S100 | 6 |
| Ethylcellulose 100 | 1 |
| Span ® 20 | 2.8 |
| Propylene Glycol | 25 |
| Ethanol abs. | 65 |

Preparation

In a well-covered vessel, Ethylcellulose was dispersed slowly with mixing in half of the quantity of ethanol and mixed to dissolve. In another well-covered vessel, Eudragit* was slowly dispersed with mixing in the other half of the quantity of ethanol and mixed to dissolve. The both solutions are then mixed together. Span® 20 was added with mixing and then Propylene glycol was added with mixing. Mitomycin C was then added with mixing. The final formulation was mixed.

### Example 48

| | % w/w |
|---|---|
| Adriamycin | 0.2 |
| Eudragit ® S100 | 6.8 |
| Phospholipid | 6 |
| Propylene Glycol | 40 |
| Ethanol 95 | 47 |

Preparation

In a well-covered vessel, phospholipid was added to ethanol and mixed to dissolve. Eudragit® was then added to ethanol and mixed until dissolved. Propylene glycol was added with mixing. Adriamycin was then added with mixing. The final formulation was mixed.

### Example 49

| | % w/w |
|---|---|
| Erytromycin ethylsuccinate | 2 |
| Eudragit ® S100 | 7 |
| Phospholipon ® 90G | 7 |
| Propylene Glycol | 44 |
| Ethanol abs. | 40 |

Preparation

In a well-covered vessel, phospholipid was added to ethanol and mixed to dissolve. Eudragit® S100 was then slowly added to ethanol and mixed until dissolved. Propylene glycol was added with mixing. Erythromycin ethylsuccinate was then added with mixing. The final formulation was mixed.

### Example 50

| | % w/w |
|---|---|
| Fentanyl | 0.005 |
| Eudragit ® S100 | 7 |
| Eudragit ® L100 | 2 |
| Ethanol abs. | 12 |
| Span ® 20 | 2 |
| Propylene Glycol | 76.995 |

Preparation

In a well-covered vessel, Eudragits® are added to the mixture of ethanol and propylene glycol and mixed to dissolve. Fentanyl was then added. Span® 20 was added and mixed. The final formulation was mixed.

### Example 51

| | % w/w |
|---|---|
| Fentanyl | 0.01 |
| Eudragit ® S100 | 6 |
| Eudragit ® RS 100 | 2 |
| Ethanol abs. | 12 |
| Span ® 20 | 1 |
| Propylene Glycol | 78.99 |

Preparation

In a well-covered vessel, Eudragits® are added to the mixture of ethanol and propylene glycol and mixed to dissolve. Fentanyl was then added. Span® 20 was added and mixed. The final formulation was mixed.

### Example 52

| | % w/w |
|---|---|
| Oxybutynin | 0.1 |
| Eudragit ® RS100 | 2 |
| Eudragit ® S100 | 7 |
| Span ® 20 | 5 |
| Propylene Glycol | 86.9 |

Preparation

In a well-covered vessel, Eudragits® are added to propylene glycol and mixed to dissolve. Span® 20 was added and mixed. Oxybutynin was then added. The final formulation was mixed.

### Example 53

| | % w/w |
|---|---|
| Diclofenac sodium | 10 |
| Eudragit ® S100 | 6 |
| Polycarbophil | 2 |
| Span ® 20 | 5 |
| Propylene glycol | 65 |
| Ethanol 96 | 12 |

Preparation

In a well-covered vessel, Eudragit® and Polycarbophil were added to ethanol and propylene glycol mixture and mixed. Span® 20 was added and mixed. Diclofenac sodium was then added to the solution with mixing. The final formulation was mixed.

### Example 54

| | % w/w |
|---|---|
| Tramadol HCl | 10 |
| Eudragit ® RL100 | 6 |
| Eudragit ® RS 100 | 2 |
| Ethanol 95 | 50 |
| Polysorbate 80 | 5 |
| Propylene Glycol | 27 |

Preparation

In a well-covered vessel, Eudragits® are added to the mixture of ethanol and propylene glycol and mixed. Tramadol was then added. Polysorbate was added and mixed. The final formulation was mixed.

### Example 55

| | % w/w |
|---|---|
| Ibuprofen | 10 |
| Eudragit ® E100 | 6 |
| PLGA | 2 |
| Ethanol abs. | 50 |
| Polysorbate 80 | 5 |
| Propylene Glycol | 27 |

Preparation

In a well-covered vessel, Eudragit® was added to the mixture of ethanol and propylene glycol and mixed. Polysorbate was added and mixed. PLG was added and mixed. Ibuprofen was added and mixed. The final formulation was mixed.

### Example 56

| | % w/w |
|---|---|
| Ibuprofen | 5 |
| Eudragit ® S100 | 7 |
| PVA | 2 |
| Ethanol abs. | 50 |
| Span ® 20 | 5 |
| Propylene Glycol | 31 |

Preparation

In a well-covered vessel, Eudragit® was added to the mixture of ethanol and propylene glycol and mixed. Span® was added and mixed. PLG was added and mixed. Ibuprofen was added and mixed. The final formulation was mixed.

### Example 57

| | % w/w |
|---|---|
| Ketoprofen | 8 |
| Eudragit ® S100 | 8 |
| Triacetin | 1 |
| Ethanol 96 | 20 |
| Span ® 20 | 5 |
| Propylene Glycol | 58 |

Preparation

In a well-covered vessel, Eudragit® was added to the mixture of ethanol and propylene glycol and mixed. Span® was added and mixed. Triacetin was added and mixed. Ketoprofen was added and mixed. The final formulation was mixed.

### Example 58

| | % w/w |
|---|---|
| Fentanyl | 0.01 |
| Eudragit ® S100 | 6 |
| Span ® 20 | 6 |
| Propylene Glycol | 77.99 |

Preparation

In a well-covered vessel, Eudragit® was added to propylene glycol and mixed to dissolve. Span® was added and mixed. Fentanyl was then added and mixed. The final formulation was mixed.

### Example 59

| | % w/w |
|---|---|
| Cannabidiol | 0.5 |
| Oxybutynin | 0.5 |
| Ethylcellulose 100 | 7 |
| Span ® 20 | 2 |
| Ethanol abs. | 90 |

Preparation

In a well-covered vessel, Ethylcellulose was added to ethanol and mixed. Oxybutinin and Cannabidiol are then added to the polymer solution with mixing. Span® 20 was added and mixed. The final formulation was mixed.

### Example 60

| | % w/w |
|---|---|
| Cannabidiol | 0.5 |
| Lidocaine | 1 |
| Eudragit ® S100 | 8 |
| Span ® 20 | 2 |
| Propylene glycol | 38.5 |
| Ethanol abs. | 50 |

Preparation

In a well-covered vessel, Eudragit® was added to ethanol and mixed. Cannabidiol and Lidocaine were then added to the polymer solution with mixing. Span® 20 was added and mixed. Finally, propylene glycol was added. The final formulation was mixed.

### Example 61

| | % w/w |
|---|---|
| Mirabegron | 0.2 |
| Eudragit ® S100 | 7.8 |
| Span ® 20 | 3 |
| Propylene Glycol | 44 |
| Ethanol 96 | 45 |

Preparation

In a well-covered vessel, Mirabegron was added to ethanol and mixed. Eudragit® was added with continues mixing until a clear solution was obtained. The final formulation was mixed.

### Example 62

| | % w/w |
|---|---|
| Tramadol HCl | 1 |
| Eudragit ® S100 | 7 |
| Propylene Glycol | 42 |
| Ethanol 95 | 50 |

Preparation

In a well-covered vessel, Eudragit® was added to ethanol 95% and mixed until a solution was received, propylene glycol was added and mixed, then Tramadol was added and mixed.

One ml of the formulation was injected into 100 ml simulated urine fluid. The capture mass was instantaneously formed. The capture mass had a largest dimension of about 4 cm.

### Example 63

| | % w/w |
|---|---|
| Tramadol HCl | 1 |
| Eudragit ® S100 | 7 |
| Propylene Glycol | 52 |
| Ethanol 90 | 40 |

Preparation

In a well-covered vessel, Eudragit® was added to ethanol 90% and mixed until a clear solution was obtained, propylene glycol was added and mixed, then Tramadol was added and mixed.

One ml of the formulation was injected into 100 ml simulated urine fluid. The capture mass was instantaneously formed. The capture mass had a largest dimension of about 4 cm.

### Example 64

| | % w/w |
|---|---|
| Tramadol HCl | 1 |
| Eudragit ® S100 | 6 |
| Propylene Glycol | 31 |
| Ethanol 90 | 62 |

Preparation

In a well-covered vessel, Eudragit® was added to ethanol 90% and mixed until a clear solution was obtained, propylene glycol was added and mixed, then Tramadol was added and mixed.

1 ml of the formulation was injected into 100 ml simulated urine fluid. The capture mass was instantaneously formed. The capture mass had a largest dimension of about 4 cm.

### Example 65

| | % w/w |
|---|---|
| Tramadol HCl | 1 |
| Eudragit ® S100 | 7 |
| Propylene Glycol | 42 |
| Ethanol 96 | 50 |

Preparation

In a well-covered vessel, Eudragit® was added to ethanol 96% and mixed until a clear solution was obtained, propylene glycol was added and mixed, then Tramadol was added and mixed.

One ml of the formulation was injected into 100 ml simulated urine fluid. The capture mass was instantaneously formed. The capture mass had a largest dimension of about 4 cm.

### Example 66

| | % w/w |
|---|---|
| Oxybutynin | 0.1 |
| Eudragit ® L100 | 8 |
| Propylene Glycol | 41.9 |
| Ethanol abs. | 50 |

Preparation

In a well-covered vessel, 50% of the Oxybutynin amount was added to ethanol and mixed. Eudragit® L100 was then added until a clear solution is received, propylene glycol was added and mixed, then the rest of the Oxybutynin was added and mixed well.

### Example 67

| | % w/w |
|---|---|
| Oxybutynin | 0.1 |
| Eudragit ® L100 | 6 |
| Ethylcellulose | 3 |
| Ethanol 95 | 91.9 |

Preparation

In a well-covered vessel, Ethylcellulose was added to ethanol and mixed, then 50% of the Oxybutynin were added and mixed. Eudragit® L100 was then added until a clear solution was formed, then the rest of the Oxybutynin was added and mixed well.

### Example 68

| | % w/w |
|---|---|
| Oxybutynin | 0.4 |
| Eudragit ® L100 | 8 |
| Polysorbate 80 | 3 |
| Tromethamine | 3 |
| Propylene Glycol | 43.6 |
| Ethanol 95 | 42 |

Preparation

In a well-covered vessel, Eudragit® L100 was added to Ethanol and mixed, then Polysorbate 80 and Tromethamine were added with mixing. Propylene glycol and Oxybutynin were added and mixed.

### Example 69

| | % w/w |
|---|---|
| Oxybutynin | 0.5 |
| Lidocaine HCl | 2 |
| Eudragit ® S100 | 7 |
| Polysorbate 80 | 4 |
| Propylene Glycol | 40.5 |
| Ethanol 95 | 46 |

Preparation

In a well-covered vessel, Eudragit® S100 was added to ethanol and mixed, then polysorbate and oxybutynin were added and mixed. Lidocaine was then added with mixing. Propylene glycol was added and mixed.

### Example 70

| | % w/w |
|---|---|
| Lidocaine HCl | 2 |
| Eudragit RS100 | 7 |
| Tromethamine | 2 |
| Polysorbate 20 | 4 |
| Propylene Glycol | 40 |
| Ethanol 95 | 45 |

Preparation

In a well-covered vessel, Eudragit® RS100 was added to ethanol and mixed, then polysorbate 20 was added and mixed. Lidocaine was added with mixing. Tromethamine and propylene glycol were added with mixing. The final formulation was mixed.

### Example 71

| | % w/w |
|---|---|
| Lidocaine HCl | 1 |
| Eudragit RS100 | 8 |
| Tromethamine | 4 |
| Polysorbate 20 | 5 |
| Propylene Glycol | 42 |
| Ethanol 95 | 40 |

Preparation

The formulation was prepared according to the method described in Example 70.

### Example 72 Pre-Formulation not Containing an Active Agent

| | % w/w |
|---|---|
| Ethocer 100 | 4 |
| Carbpol 974P | 2 |
| Span 20 | 19.5 |
| Ethanol abs. | 74.5 |

Preparation

In a closed vessel, Ethocel™ 100 was added to Ethanol with mixing. Span® 20 was added and mixed. Carbpol® 974P was added, mixed, and the pre-formulation was left for about two hours and then mixed again. 3 ml of the pre-formulation were instilled in 30 ml urine pH 7.

Results

As shown in FIG. 1, the mass 10 was formed in urine 20 by precipitation of polymers. The mass 10 was floating on the surface of urine 20. The mass 10 depicted in FIG. 1 had a largest dimension of about 6 cm.

### Example 73

| | |
|---|---|
| Botulinum toxin A | 6000 units |
| Eudragit RS100 | 8% w/w |
| Tween 80 | 2% w/w |
| Benzyl alcohol | 0.5% w/w |
| Propylene glycol to | 100% w/w |

Preparation

In a well-covered vessel, Eudragit® was added to propylene glycol and mixed. Tween® 80 was added and mixed. Benzyl alcohol was added and mixed. Botulinum toxin A was added and the final formulation was mixed. The formulation was stored at 4° C.

### Example 74

Preparation

2 mg methylene blue were incorporated in 2 g of the formulation prepared according to Example 23, and injected using a syringe into 15 ml urine. A blue mass immediately formed and the urine started to change its color into a blue-greenish color. The mass was transferred over time in vials with clean urine.

Results

For 48 hours, methylene blue continually released and urine color changed. Urine was colored by the released methylene blue from the mass over time. The color of the urine was observed at various time points ranging from 3 hours to 48 hours. Each urine sample at each time point (i.e. from 3 to 48 hours) was blue, demonstrating that the formulations described herein are able to provide prolonged release of active agents. The mass remained colored at 48 hours following the beginning of the experiment, demonstrating that some of the methylene blue remained in the mass and could continue to be released into urine even after 48 hours.

### Example 75

TABLE 1
Ingredients of five different formulations.

| | Formulation (g) | | | | |
|---|---|---|---|---|---|
| Ingredient | 1 | 2 | 3 | 4 | 5 |
| Ethanol absolute | 5.0 | 5.0 | | 4.6 | 5.0 |
| Eudragit ® S100 | 0.8 | 0.8 | 0.8 | 0.4 | 0.8 |
| Eudragit ® L100 | | | | 0.4 | 0.2 |
| Propylene Glycol | 3.7 | 4.2 | 9.2 | 4.6 | 4.0 |
| Span ® 20 | 0.5 | | | | |

Preparation

In a well-covered vessel, the Eudragits® were added to ethanol and mixed, propylene glycol was added and mixed, then Span® 20 was added and mixed.

The formulations in Table 1 were examined for their appearance (results shown in Table 2) after preparation and following 1.5 ml injection to 200 ml water at room temperature (RT).

TABLE 2
Appearance of different formations and mass formed from the
different formulations.

| | | Appearance of capture | Appearance of |
|---|---|---|---|
| | | mass formed in water | capture mass after 24 |
| Formulation | Appearance | at RT after injection | hours in water at RT |
| 1 | clear | single white round | floating on water |
| | yellowish | floating solid (d 3 cm) | surface, slight signs |
| | solution | | of erosion |
| 2 | clear | single white round | immersed in water, |
| | colorless | solid (d 4 cm) | slight signs of |
| | solution | | erosion |
| 3 | clear | single white round | floating on the water |
| | colorless | solid (d 4 cm) | surface, slight signs |
| | solution | | of erosion |
| 4 | clear | spread single white | fully disintegrated |
| | colorless | round solid (d 8 cm) | |
| | solution | | |
| 5 | clear | single white round | immersed in water, |
| | colorless | solid (d 4 cm) | no signs of erosion, |
| | solution | | |

d—largest dimension
RT—room temperature

The data in Table 2 illustrate the ability to modulate the formulations to form capture masses with different properties, as demonstrated by their differences in erosion and disintegration after 24 hours in water at room temperature.

### Example 76

TABLE 3
Ingredients of five different formulations.

| | Formulation (g) | | | | |
|---|---|---|---|---|---|
| Ingredient | 1 | 2 | 3 | 4 | 5 |
| Ethanol absolute | 5.4 | 5.2 | 5.2 | 5.0 | |
| Eudragit ® S100 | 0.6 | 0.6 | 0.6 | 0.7 | 0.8 |
| Eudragit ® L100 | | 0.2 | | 0.1 | |
| Propylene Glycol | 4.0 | 4.0 | 4.0 | 3.8 | 9.2 |
| Span ® 20 | | | | 0.4 | |
| Klucel ™ | | | 0.2 | | |

Preparation

In a well-covered vessel, Eudragits® were added to ethanol and mixed, propylene glycol was added and mixed, then Span® 20 or Klucel™ were added and mixed.

The formulations in Table 3 were examined for their appearance (results shown in Table 4) after preparation and following 1.5 ml injection into 200 ml water at room temperature (RT).

TABLE 4
Appearance of different formations and mass formed from the
different formulations.

| | | Appearance of capture mass |
|---|---|---|
| | | formed in water at RT after |
| Formulation | Appearance | injection |
| 1 | clear colorless solution | single white round solid (d 4 cm) |
| 2 | clear colorless solution | single white round solid (d 5 cm) |
| | | layer |
| 3 | clear colorless solution | single white round solid (d 5 cm) |
| 4 | clear yellowish solution | single white round solid (d 4 cm) |
| 5 | clear colorless solution | single white round solid (d 4 cm) |

dlargest dimension
RTroom temperature

### Example 77 Exemplary Tramadol Formulations

TABLE 5
Ingredients in two different Tramadol formulations.

| | (g) formulation | |
|---|---|---|
| Ingredient | 1 | 2 |
| Ethanol absolute | 5.0 | 5.0 |
| Eudragit ® S100 | 0.8 | 0.8 |
| Propylene Glycol | 3.7 | 4.2 |
| Span ® 20 | 0.5 | — |
| Tramadol HCl | 0.02 | 0.02 |

Preparation

In a well-covered vessel, Eudragit® was added to ethanol and mixed. Span® (when present) was added and mixed, then propylene glycol was added and mixed. The formulations were kept at room temperature for 48 hours, after which 20 mg Tramadol were added to each formulation and mixed well.

### Example 78 Tramadol HCl Release Profile from Capture Drug Delivery Formulations—In Vitro Experiment

Objective

To evaluate the prolonged profile Tramadol release from the delivery system for instillation to bladder.

Method

In this experiment, the release of Tramadol HCl the two capture formulations described in Example 77 were tested. Each formulation contains 20 mg Tramadol HCl in 10.02 g formulation in comparison with a control solution (which contains 1:1 w/w ethanol:propylene glycol) containing 20 mg Tramadol HCl in 10.02 g solution. The presence of Tramadol in the simulated urine fluid (SUF) (Abdelrahman Y. Sherif, et al., Saudi Pharmaceutical Journal, 26(6):845-851 (2018)) was determined by using TRA single Drug Urine Test Stick (cat no. WDTR-114, Lot no WO8690402) which is a competitive binding, lateral immunochromatographic assay for qualitative simultaneous detection of Tramadol in urine at a specified cutoff of 200 ng/ml.

Procedure

The formulations were prepared according to Example 77.

One mL of each capture formulation (Formulations 1 and 2 described above in Table 5) was injected into a beaker containing 100 mL SUF. Formulation 1 was tested in two duplicates (referred to as Formulations 1a and 1b in Table 7), and Formulation 2 was tested in two duplicates (referred to as Formulation 2a and 2b in Table 7). One mL Tramadol control solution at the same drug concentration as in the delivery systems was injected into a beaker containing 100 mL SUF (control vessel). The five beakers were kept during the experiment in an orbital shaker incubator at 36.7 C, 20 rpm.

At four predetermined time points during a 22-hour period, the detection of the released Tramadol drug in SUF, at each time point, was made using the Urine dip sticks, after which the SUF was emptied from the beaker, washed twice with 100 mL SUF, and a new volume of 100 ml SUF, preheated at the experiment temperature, was introduced into each beaker. First sample from each formulation was taken immediately after the injection into the SUF.

TABLE 6
Simulated Urine Fluid (SUF)

| | g/L |
|---|---|
| NaCl | 13.75 |
| MgSO₄ | 1.69 |
| MgCL₂ | 0.83 |
| CaCl₂ | 0.67 |
| KCl₂ | 0.38 |
| Urea | 17.4 |

Results

The results presented in Table 7 show that the drug incorporated in the delivery system was present in the system up to the end of the experiment, 22 hours. At 0 h, each of formulations 1 and 2 formed a floating round white mass. The largest dimension (d) of each mass was about 4 cm. The cumulative minimum amount of Tramadol released in the urine at the 22 hours time point can be estimated, for each of the four tested formulations, to be greater than 80 μg.

The capture delivery systems were present in simulated urine fluid, showing slight erosion or no erosion, depending on the formulation, up to the last experimental point, 22 hours. Span® 20, 5% w/w in Formulation 1 of the experiment, contributed to a slight erosion of the system in urine.

In contrast to the above results obtained from the delivery systems, the drug in the control vessels was not detected (Negative) at the 1.5 h test point.

TABLE 7
Detection of Tramadol at various time points

| | Time, h | | | | | | |
|---|---|---|---|---|---|---|---|
| Formulation | 0 | 1.5 | | 5 | | 22 | |
| 1a | P | P | ED1 | P | ED1 | P | ED2 |
| 1b | P | P | ED1 | P | ED1 | P | ED2 |
| 2a | P | P | ED1 | P | ED1 | P | ED1 |
| 2b | P | P | ED1 | P | ED1 | P | ED1 |
| Control | — | N | — | N | — | N | — |

N—Negative presence of Tramadol <200 ng/ml Tramadol
P—Positive presence of Tramadol >200 ng/ml Tramadol

TABLE 8
Erosion and disintegration key for results in Table 7

| | Erosion/Disintegration rate |
|---|---|
| ED1 | no signs of erosion or disintegration |
| ED2 | slight peripheral erosion |

Conclusion

The formulations instilled in simulated urine fluid formed a mass, which is a delivery system, in situ, releasing Tramadol for at least 22 hours, from the time of the mass formation. The system integrity could be altered by the formulation components, thereby altering the release profile of the active agent.

### Example 79. Oxybutynin HCL Release Profile from Capture Drug Delivery System—Ex Vivo Experiment to be Performed in Pig Isolated Bladder

Objective

To evaluate the release profile of Oxybutynin from a delivery system formulation for instillation to bladder.

TABLE 9
Ingredients in the Formulations to be tested

| | (g) formulation | |
|---|---|---|
| | Test | Control |
| | Formulation | Formulation |
| Ethanol absolute | 9.8 | 10.0 |
| Eudragit ® L100 | 1.4 | — |
| Propylene Glycol | 8.4 | 9.6 |
| Oxybutynin HCl | 0.4 | 0.4 |

Preparation

In a well-covered glass vile, Oxybutynin HCL and ethanol are mixed until a clear solution is obtained. Eudragit® (if present) is added to the formulation and mixed well, then propylene glycol is added and mixed well.

Protocol

In this experiment, a modified ex vivo method presented by Gross et al. the Journal of Urology, 183(4):e74-e75 (2010) will be used.
a. 6 fresh excised whole pig bladders including urethra and ureters, will be supplied from a food slaughter place. Each urethra will be catheterized with a silicon tube. The ureters will be catheterized with luer female connector secured with silk thread and connected through a Y connector assembly to silicon tubes.
b. Each bladder will be immersed in bath filled with Krebs solution at 37° C.
c. Simulated urine fluid (SUF) (Abdelrahman Y. Sherif, et al., Saudi Pharmaceutical Journal, 26(6):845-851 (2018)) will be the release medium in the bladder for Oxybutynin from the delivery system. Each bladder will be prefilled with 50 ml SUF.
d. The filling rate of the SUF into the bladder will have a constant filling cycle of 2 ml/minute during 16 hours and of 1 ml/minute during 8 hours.
e. A measured volume of 2 ml from the Test formulation containing 2% w/w Oxybutynin will be instilled into each one of the 3 bladders. The same volume of the control formulation, at a same drug concentration, will be instilled into each one of other 3 bladder.
f. The medium will be voided at seven predetermined time points: 0, 4, 8, 12, 16, 24, and 28 hours, leaving 50 ml of SUF in the bladder, in order to resemble urine remnants after urination in humans.
g. At seven predetermined time points: 0, 4, 8, 12, 16, 24 and 28 hours, 5 ml of SUF from each bladder will be collected and stored in −18° C. for quantitative analysis of Oxybutynin by HPLC.

### Example 80. Tramadol HCl Release Profile from Three Formulations—In Vitro Experiment

Objective

To evaluate the prolonged profile Tramadol release from the new delivery system for instillation to bladder.

Method

In this experiment, the release of Tramadol HCl from three formulations were tested. Each formulation contained 20 mg Tramadol HCl (about 0.2% w/w of the formulation) in comparison with a control solution (which contained 1:1 w/w ethanol:propylene glycol and Tramadol HCl) containing the same drug dose (20 mg Tramadol HCl). The presence of Tramadol in SUF (Abdelrahman Y. Sherif, et al., Saudi Pharmaceutical Journal, 26(6): 845-851 (2018)) was determined by using TRA single Drug Urine Test Stick (cat no. WDTR-114, Lot no WO8690402) which is a competitive binding, lateral immunochromatographic assay for qualitative simultaneous detection of Tramadol in urine at a specified cutoff of 200 ng/ml.

Procedure

In a well-covered vessel, Eudragits® were added to ethanol and mixed. Klucel® (if present) was added and mixed then propylene glycol was added and mixed. Finally, Tramadol was added and the final formulation was mixed.

1.5 mL of each capture formulation described in Table 10 was injected into a beaker containing 100 mL SUF prepared as described in Table 6. Formulations 1, 2, 3 listed in Table 10 were each tested in two duplicates (1a, 1b, 2a, 2b, 3a and 3b).

1.5 mL Tramadol control solution (control) at the same drug concentration as in the formulations listed in Table 10 was injected into a beaker containing 100 mL SUF (control vessel).

The seven beakers were kept during the experiment in an orbital shaker incubator at 36.7° C., 20 rpm.

At six predetermined time points during a 24-hour period, the detection of the released Tramadol drug in SUF, at each time point, was made using the Urine dip sticks, after which the SUF was emptied from the beaker, washed twice with 100 mL SUF, and a new volume of 100 ml SUF, preheated at the experiment temperature (36.7° C.), was introduced into each beaker.

The first sample from each formulation was taken immediately (i.e. time 0 hours) after the injection into the SUF.

The degree of erosion and or disintegration at each time point were noted and are defined using the key provided in Table 12.

TABLE 10
Capture formulations

| | (g) formulation | | |
|---|---|---|---|
| Ingredient | 1 | 2 | 3 |
| Ethanol absolute | 5.0 | 5.2 | 5.2 |
| Propylene Glycol | 4 | 4 | 4 |
| Eudragit ® S100 | 0.8 | 0.6 | 0.6 |
| Eudragit ® L100 | 0.2 | 0.2 | — |
| Klucel ™ | — | — | 0.2 |
| Tramadol HCl | 0.02 | 0.02 | 0.02 |

Results

The results presented in Table 11 show that the drug incorporated in the delivery system was present in the system up to the end of the experiment, 24 hours. At 0 h, each of formulations 1, 2, and 3 formed a floating round white mass. The largest dimension (d) of each mass was about 7 cm.

The cumulative minimum amount of Tramadol released in the urine at the 24 hour time point can be estimated, for each formulation 1 and 2 of the tested formulations, to be greater than 120 μg, and for formulation 3 to be greater than 100 μg.

The capture mass formed from each formulation was present in SUF, showing various degrees of erosion and disintegration, depending on the formulation, up to the last experimental point, 24 hours. Klucel™ 2% w/w in Formulation 3 of the experiment, contributed to the disintegration of the mass in the SUE.

In contrast to the above results obtained from the delivery systems, the drug was not detected in the control vessel (Negative) at the 2 h test point, i.e. at 2 hours or later after placement in SUE.

TABLE 11
Detection of Tramadol at various time points and “capture” mass
appearance

| | | Time, h | | | | | | | | | | | |
|---|---|---|---|---|---|---|---|---|---|---|---|---|---|
| Formulation | | 0 | | 2 | | 4 | | 8 | | 16 | | 24 | |
| 1 | a | P | d7 cm | P | ED1 | P | ED1 | P | ED2 | P | ED3 | P | ED3 |
| | b | P | d7 cm | P | ED1 | P | ED1 | P | ED2 | P | ED3 | P | ED3 |
| 2 | a | P | d7 cm | P | ED1 | P | ED1 | P | ED3 | P | ED3 | P | ED4 |
| | b | P | d7 cm | P | ED1 | P | ED1 | P | ED3 | P | ED3 | P | ED4 |
| 3 | a | P | d6 cm | P | ED1 | P | ED3 | P | ED4 | P | ED4 | N | ED5 |
| | b | P | d7 cm | P | ED1 | P | ED3 | P | ED4 | P | ED4 | N | ED5 |
| Control | | P | | N | — | — | — | — | — | — | — | — | — |

N—Negative presence of Tramadol <200 ng/ml Tramadol
P—Positive presence of Tramadol >200 ng/ml Tramadol

TABLE 12
Erosion and disintegration key for results in Table 11

| | Erosion/Disintegration rate |
|---|---|
| ED1 | no signs of erosion or disintegration |
| ED2 | slight peripheral erosion |
| ED3 | marked erosion |
| ED4 | erosion + slight disintegration (presence of particles > 2-3 mm) |
| ED5 | full disintegration > particles 2-3 mm |
| ED6 | full disintegration < particles 2-3 mm |

Conclusion

The formulations instilled in SUF formed a mass in situ, which serves as a delivery system, in situ, releasing Tramadol for at least 16 hours (Formulation 3) and at least 24 hours (Formulations 1 and 2), from the time of the mass formation (i.e. initial placement in SUF). As demonstrated by the different appearances of the mass formed from Formulation 3 compared to the masses formed from Formulations 1 and 2 at the 24 hour time point, the erosion or degradation of a mass can be altered by varying the components in the formulation and thereby altering the release profile of the active agent.

### Example 81. In Vivo Experiment: Evaluation of Oxybutynin HCL Release Profile from Capture Mass

Objectives

To evaluate Oxybutynin levels in plasma and urine of swine in vivo for 24 hours following instillation of a capture formulation. To evaluate the release profile of Oxybutynin HCL during a 24 hour-long experiment based on the obtained data.

Study Groups

Group 1: Three female domestic swine are treated with 3 ml formulation.

Group 2: Two Female domestic swine are treated with a control formulation of a same volume and drug concertation.

Materials and Methods

All procedures performed on animals are according to The National Institutes of Health regulations and approved by the Committee for Animal Care and Experimental Use and Ethics.

Female domestic swine weighting 60-65 kg are climatized within the animal facility for 5 days before the study. The animals are anesthetized under general anesthesia. Blood samples are taken before the study. Intravesical administration of the Test formulation and Control is performed using a syringe connected to catheter. Blood and urine sampling will be taken at time points: 0, 8, 16, 20, and 24 hours. Animal urine volume will be recorded.

At the end of the study, the animals will be euthanized. Samples will be stored in −18° C. for analytical quantitative analysis of Oxybutynin.

### Example 82: Formulation to be Made

| | % w/w |
|---|---|
| Active Agent | 0.05 to 5 |
| Eudragit ® S100 | 8 |
| Tween ® 80 | 6 |
| Ethanol 95 | 12 |
| Propylene glycol | to 100_{[DT7]} |

Preparation

In a well-covered vessel, Eudragit® is added to propylene glycol and mixed. Ethanol is added and mixed. Tween® 80 is added and mixed. The active agent is added and mixed.

1.-86. (canceled)

87. A liquid formulation for instillation into the urinary tract, bladder, or kidney(s) comprising:
an alcohol,
one or more polymers wherein the one or more polymers are soluble in the alcohol and practically insoluble in water or an aqueous solution at a pH≤6; and
one or more active agents,
wherein the formulation is forming upon instillation a mass in the urinary tract, bladder, or kidney(s), and wherein the mass releases the one or more active agents over an extended period of time.

88. The formulation of claim 87, wherein the alcohol is a short-chain alcohol.

89. The formulation of claim 88, wherein the alcohol is a C2-C4 alcohol.

90. The formulation of claim 88, wherein the alcohol is ethanol or propylene glycol.

91. The formulation of claim 87, wherein the one or more polymers comprise an acrylate polymer, and wherein the acrylate polymer is poly(methacrylate), poly(ethylacrylate), a copolymer thereof, or polycarbophil, poly(methyl methacrylate), poly(ethyl methacrylate), poly(N,N-dimethylaminoethyl methacrylate), poly(butyl methacrylate), poly(ethylacrylate), Eudragit®, Eudragit S100® or Eudragit L100®.

92. The formulation of claim 87, further comprising one or more additives, wherein the one or more additives comprise:
molecules, oligomers, and/or polymers that are water- and alcohol-dispersible,
one or more surfactant and phospholipids,
one or more pharmaceutically acceptable excipients selected from the group consisting of plasticizers, viscosity modifiers, a pH buffering agent, permeation enhancers, diluents, lubricants, preservatives, antioxidants, binders, disintegrators, and stabilizers, or a combination thereof.

93. The formulation of claim 87, wherein a total concentration of the one or more polymers is in a range from 1% to 20% w/w, from 2% to 20% w/w, from 1% to 10% w/w, from 2% to 8% w/w, or from 2% to 7% w/w of the formulation.

94. The formulation of claim 87, wherein the total concentration of the alcohol is from 10% to 90% w/w, from 12% to 50% w/w, from 20% to 80% w/w, from 30% to 75% w/w, from 40% to 90% w/w, from 50% to 95% w/w, or from 60% to 98% w/w of the formulation.

95. The formulation of claim 87, wherein the polymers are selected from Eudragit, Eudragit S100® or/and Eudragit L100® and wherein the alcohol is a C2-C4 alcohol, such as ethanol or propylene glycol.

96. A pre-filled syringe or container comprising the formulation of claim 87.

97. A method for treating or ameliorating one or more symptoms associated with a disorder or disease in or affecting a patient's urinary tract, bladder, and/or kidney(s) comprising:
(i) inserting a catheter, a cystoscope, or an ureteroscope,
(ii) instilling the formulation of claim 87 into the urinary tract, bladder, and/or kidney(s) through the catheter, the cystoscope, or the ureteroscope, into the urinary tract, bladder, and/or kidney(s) of the patient, and
wherein following step (ii), the formulation forms a mass and the one or more active agents are entrapped in the mass.

98. The method of claim 97, wherein the formulation has an instillation volume from 0.5 mL to 120 mL, from 4 mL to 100 mL, from 2 mL to 60 mL, from 5 mL to 40 mL, from 1 mL to 30 mL, from 1 mL to 10 mL, or from 1 mL to 5 mL.

99. The method of claim 97, further comprising repeating step (ii) with the same formulation or a different formulation at determined time.

100. The method of claim 97, wherein step (ii) is repeated

101. The method of claim 97, further comprising step (iii) adjusting the release rate of the one or more active agents from the mass prior to step (i), subsequent to step (i) and prior to step (ii) and/or subsequent to step (ii) by instilling a basic solution into the urinary tract, bladder, and/or kidney(s), administering bicarbonate medications, or consuming food associated with raising the urine pH, or a combination thereof.

102. The method of claim 97, wherein the patient has one or more symptoms associated with inflammation, overactive bladder and/or neurogenic bladder, pain, infection, interstitial cystitis or cancer, of the urinary tract, bladder, and/or kidney(s), and wherein the active agent is in an effective amount to treat or ameliorate at least one of the one or more symptoms.

103. The method of claim 97, wherein the formulation provides prolonged release of the one or more active agents compared to a control, wherein the control comprises the one or more active agents at the same concentration (w/w) in the same alcohol as the formulation.

104. A method for providing prolonged release of an active agent into a patient's urinary tract, bladder, and/or kidney(s) comprising:
(i) inserting a catheter, a cystoscope, or an ureteroscope into the urinary tract, bladder, and/or kidney(s) of the patient, and
(ii) instilling the formulation of claim 87 into the urinary tract, bladder, and/or kidney(s) through the catheter, the cystoscope, or the ureteroscope, wherein following step (ii), the formulation forms a mass and the one or more active agents are entrapped in the mass.

105. The method of claim 104, wherein the formulation has an instillation volume from 0.5 mL to 120 mL, from 4 mL to 100 mL, from 2 mL to 60 mL, from 5 mL to 40 mL, from 1 mL to 30 mL, from 1 mL to 10 mL, or from 1 mL to 5 mL.

106. The method of claim 104, further comprising repeating step (ii) with the same formulation or a different formulation at determined time.

107. The method of claim 104, wherein step (ii) is repeated.

108. The method of claim 104, further comprising step (iii) adjusting the release rate of the one or more active agents from the mass prior to step (i), subsequent to step (i) and prior to step (ii) and/or subsequent to step (ii) by instilling a basic solution into the urinary tract, bladder, and/or kidney(s), administering bicarbonate medications, or consuming food associated with raising the urine pH, or a combination thereof.

109. The method of claim 104, wherein the patient has one or more symptoms associated with inflammation, overactive bladder and/or neurogenic bladder, pain, infection, interstitial cystitis or cancer, of the urinary tract, bladder, and/or kidney(s), and wherein the active agent is in an effective amount to treat or ameliorate at least one of the one or more symptoms.

110. The method of claim 104, wherein the formulation provides prolonged release of the one or more active agents compared to a control, wherein the control comprises the one or more active agents at the same concentration (w/w) in the same alcohol as the formulation.
